SEQUENCE LISTING (23 sequences)

SEQ ID NO: 1 - protein, 5 aa, Artificial Sequence
  Synthetic Construct
  MISC_FEATURE (3)..(3): Xaa is any amino acid
LPXTG

SEQ ID NO: 2 - protein, 4 aa, Artificial Sequence
  Synthetic Construct
  MISC_FEATURE (1)..(1): Xaa is any amino acid except Pro, Asp, or Glu
  MISC_FEATURE (2)..(2): Xaa is Ile, Leu, Val, or Cys
  MISC_FEATURE (3)..(4): Xaa is any amino acid
XXXX

SEQ ID NO: 3 - protein, 4 aa, Artificial Sequence
  Synthetic Construct
  MISC_FEATURE (3)..(4): Xaa is any amino acid
RHXX

SEQ ID NO: 4 - protein, 5 aa, Artificial Sequence
  Synthetic Construct
  MISC_FEATURE (2)..(2): Xaa is any amino acid
  MISC_FEATURE (4)..(4): Xaa is any amino acid
CXPXR

SEQ ID NO: 5 - protein, 5 aa, Artificial Sequence
  Synthetic Construct
LLQGA

SEQ ID NO: 6 - protein, 12 aa, Artificial Sequence
  Synthetic Construct
VDSVEGEEEGEE

SEQ ID NO: 7 - protein, 11 aa, Artificial Sequence
  Synthetic Construct
DSLEFIASKLA

SEQ ID NO: 8 - protein, 10 aa, Artificial Sequence
  Synthetic Construct
ATHIKFSKRD

SEQ ID NO: 9 - protein, 13 aa, Artificial Sequence
  Synthetic Construct
AHIVMVDAYKPTK

SEQ ID NO: 10 - protein, 23 aa, Artificial Sequence
  Synthetic Construct
DIPATYEFTDGKHYITNEPIPPK

SEQ ID NO: 11 - protein, 12 aa, Artificial Sequence
  Synthetic Construct
KLGSIEFIKVNK

SEQ ID NO: 12 - DNA, 13 bp, Artificial Sequence
  Synthetic Construct
tgcaagtcatcgg

SEQ ID NO: 13 - protein, 24 aa, Artificial Sequence
  Synthetic Construct
DPAALKRARNTEAARRSRARKGGC

SEQ ID NO: 14 - DNA, 314 bp, Artificial Sequence
  Synthetic Construct
gggctactactataatacgactcactatagggtaagtgtggaaggagatatacatatggattataaattagatgatggcgattacaagctcgacgatattgactataaactggatgacgacaagggttccggaagttacccttatgatgtgcctgactatgccggatctggcagtgattataaactcgatgatggagactataaattagacgacatcgactataaactggacgacgacaaggggtccggctcgttacctgaaacaggatgatgagcgggccgcagggttttttgctgccgtatgactcatatgc

SEQ ID NO: 15 - DNA, 314 bp, Artificial Sequence
  Synthetic Construct
gggctactactataatacgactcactatagggtaagtgtggaaggagatatacatatggattataaagatgaagatggagactacaaagacgaagacattgactacaaagacgaggaccttctcgggagtggttcttatccttacgatgtgcccgactacgccgggagcggctcagattacaaagatgaggacggagattacaaagatgaagatattgactataaagacgaagatctcttagggtccggctcgttacctgaaacaggatgatgagcgagccgcagggttttttgctgccgtatgactcatatgc

SEQ ID NO: 16 - DNA, 314 bp, Artificial Sequence
  Synthetic Construct
gggctactactataatacgactcactatagggtaagtgtggaaggagatatacatatggattataaagatcatgatggtgattacaaggaccatgatatcgactataaagacgacgacgacaagggatcgggtagctatccatatgacgtgccggactatgctggatcaggcagtgactataaagaccacgatggcgactacaaagaccacgacatcgattacaaagacgacgacgataaagggtccggctcgttacctgaaacaggatgatgagcgcgccgcagggttttttgctgccgtatgactcatatgc

SEQ ID NO: 17 - DNA, 743 bp, Artificial Sequence
  Synthetic Construct
gggctactactataatacgactcactatagggtaagtgtggaaggagatatacatatgaagaagtggaccaaccgtctgatgacgatcgctggtgtggtactgatcctggtagcagcatatctgttcgctaaaccacatatcgataactacctgcacgataaagataaggatgaaaagatcgaacaatacgataaaaacgtaaaggaacaggcaagtaaagataaaaagcagcaggctaagcctcaaatcccgaaagacaagtcgaaagtggcaggttacatcgaaatcccagatgctgatatcaaagaaccagtatacccaggtccagcaacgcctgaacaactgaatcgtggtgtaagcttcgcagaagaaaacgaaagtctggatgatcaaaatattagcattgcaggccacactttcattgaccgtccgaactatcaatttacaaatctgaaagcagcaaagaaaggtagtatggtgtacttcaaagttggtaatgaaacacgtaagtataaaatgaccagcattcgtgatgttaaacctacagatgttggtgttctggatgaacaaaagggtaaagataaacaactgacactgatcacttgtgatgattacaatgaaaagacaggtgtatgggaaaaacgtaagatcttcgtggcaaccgaggtcaagtgatagcataaccccttggggcctctaaacgggtcttgaggggttttttgctgccgtatgactcatatgc

SEQ ID NO: 18 - DNA, 32 bp, Artificial Sequence
  Synthetic Construct
gggctactactataatacgactcactataggg

SEQ ID NO: 19 - DNA, 32 bp, Artificial Sequence; identical to SEQ ID NO: 18
  Synthetic Construct

SEQ ID NO: 20 - DNA, 36 bp, Artificial Sequence
  Synthetic Construct
gcatatgagtcatacggcagcaaaaaaccctgcggc

SEQ ID NO: 21 - DNA, 28 bp, Artificial Sequence
  Synthetic Construct
gcatatgagtcatacggcagcaaaaaac

SEQ ID NO: 22 - DNA, 36 bp, Artificial Sequence; identical to SEQ ID NO: 20
  Synthetic Construct

SEQ ID NO: 23 - DNA, 34 bp, Artificial Sequence
  Synthetic Construct
gctcatcatcctgtttcaggtaacgagccggacc

(19) United States
(12) Patent Application Publication
(10) Pub. No.: US 2023/0287490 A1
(43) Pub. Date: Sep. 14, 2023

(54) SYSTEMS AND METHODS FOR ASSAYING A PLURALITY OF POLYPEPTIDES

(71) Applicant: Protillion Biosciences, Inc., Burlingame, CA (US)

(72) Inventors: Michael Roy GOTRIK, Redwood City, CA (US); Curtis James LAYTON, San Bruno, CA (US); Pavanapuresan Pushpagiri VAIDYANATHAN, Redwood City, CA (US)

(21) Appl. No.: 18/007,032

(22) PCT Filed: Jul. 27, 2021
(86) PCT No.: PCT/US2021/043297

§ 371 Date: Jan. 26, 2023

Related U.S. Application Data
(60) Provisional application No. 63/057,754, filed on Jul. 28, 2020.

(51) Int. Cl. C12Q 1/6874; G01N 33/68

(52) U.S. Cl. CPC C12Q 1/6874; G01N 33/6818; G01N 2333/912

(57) ABSTRACT

The disclosure provides compositions and methods for assaying the function or properties of a plurality of polypeptides. In particular, the disclosure provides methods for high-throughput characterization of large population of polypeptides. Each polypeptide is displayed on a solid surface, such as a bead, where the solid surface also displays a nucleic acid that encodes the polypeptide. For example, each polypeptide may be covalently linked to a nucleic acid that encodes the polypeptide. In preferred embodiments, the polypeptide and nucleic acid are assayed in parallel, and with the same instrument.

### CROSS-REFERENCE TO RELATED APPLICATIONS

This application claims the benefit of U.S. Provisional Patent Application No. 63/057,754 filed Jul. 28, 2020; the disclosure of which is hereby incorporated herein by reference in its entirety.

### SEQUENCE LISTING

The instant application contains a Sequence Listing which has been submitted electronically in ASCII format and is hereby incorporated by reference in its entirety. The ASCII copy, created on Jul. 13, 2020, is named 51351-005001_Sequence_Listing_7_13_20_ST25 and is 7,496 bytes in size.

### BACKGROUND OF THE INVENTION

Directed Evolution (DE) is currently the only systematic and reliable approach for engineering novel proteins with desired properties (e.g., size, stability, folding efficiency) and/or function (e.g., binding affinity, specificity, enzymatic activity). Starting from large candidate libraries of biomolecules, DE mimics the process of natural selection to identify or evolve functional proteins and other biomolecules according to specific user-defined goals through, usually iterative, rounds of selection. However, similarly enriched biomolecules identified through DE can vary greatly in their properties, and therefore molecules identified through DE still typically need additional functional characterization using low-throughput quantitative methods. Furthermore, DE can be laborious and highly nuanced in practice, and can require weeks of work by highly skilled practitioners to produce acceptable results.

High-throughput DNA sequencing methods and instrumentation can sequence large libraries of DNA in parallel on micron to sub-micron DNA features (e.g., beads or polonies on an array) on automated instrumentation. One approach to automated, massively parallel protein functional characterization is to develop methods and compositions whereby proteins are co-localized with DNA encoding their identity such that the same automated instrumentation used to sequence the DNA is also used to measure protein biophysical properties (e.g., binding affinity) on the same bead. Furthermore, in order to perform protein assays in wide-ranging environmental conditions (pH, temperature, salt or chemical denaturant concentration, etc.), it is desirable that such DNA/protein display methods use robust covalent linkages instead of non-covalent interactions.

Therefore, there is an unmet need for compositions and methods that allow quantitative high-throughput characterization of large libraries of biomolecules. There is also a need for methods that are faster, more efficient, and more automated than DE.

### SUMMARY OF THE INVENTION

The disclosure provides compositions and methods for assaying the function and/or properties of a plurality of polypeptides. In particular, the disclosure provides methods for quantitative high-throughput characterization of a large population of polypeptides. Methods described herein are faster, more efficient, and/or allow for increased automation of directed evolution and characterization of a library of polypeptides.

The compositions and methods of the present disclosure are based, at least in part, on methods for linking a genotype (e.g., a nucleic acid, such as DNA or RNA) with an encoded phenotype (e.g., polypeptide) in a manner that is both high-throughput and compatible with automated assays performed at massive scale. In particular embodiments, the present compositions and methods link a nucleic acid with its respective encoded polypeptide on a per-bead basis, where sequencing the nucleic acid is used to reliably identify the polypeptide displayed on the bead. Furthermore, the described methods allow for the display of enough copies of the nucleic acid per bead to provide enough signal for nucleic acid sequencing and identification of the encoded polypeptide. Additionally, the described methods allow the display of enough polypeptide molecules per bead to provide sufficient signal for protein functional assays. In some embodiments, identification of the nucleic acid by sequencing and one or more functional assays of the corresponding polypeptide are performed on the bead-based library in the same instrument enabling high throughput and efficiency in the functional characterization of a large library of polypeptides.

In some embodiments of the compositions and methods described herein, each polypeptide is displayed on a solid surface, such as a bead, and the solid surface also displays a nucleic acid that encodes the identity of the polypeptide. For example, each polypeptide may be covalently linked to a nucleic acid that encodes the polypeptide, and where the nucleic acid is itself linked to the bead. In preferred embodiments, the polypeptide and nucleic acid are assayed in parallel, and with the same instrument. This enables characterization of large libraries of polypeptides. Multiple assays may be performed, in iterative rounds, on the same library of polypeptides without the need for selection, thus allowing each member to be characterized across multiple parameters in a less-costly and time-intensive manner as compared to prior art methods.

In a an aspect, the disclosure provides a method of assaying a function or property of a plurality of polypeptides. The method includes a plurality of beads, wherein each bead is conjugated to a nucleic acid molecule encoding a polypeptide, and each bead is further conjugated to the encoded polypeptide. Moreover, the method includes, in any order, the sequencing in parallel of the nucleic acid molecule conjugated to each bead to identify the polypeptide conjugated to each bead, and the assaying in parallel one or more functions or properties of each polypeptide conjugated to each bead. Furthermore, the method includes connecting the one or more functions or properties of each polypeptide to the sequence of the nucleic acid molecule encoding the polypeptide, thereby determining the identity and the one or more functions or properties of each polypeptide of the plurality of polypeptides.

In an aspect, the disclosure provides a method of high-throughput analysis of a plurality of polypeptides comprising: providing a plurality of beads, wherein a bead of the plurality of beads is conjugated to a different nucleic acid molecule encoding a polypeptide; processing the nucleic acid molecule encoding a polypeptide to produce the encoded polypeptide, wherein the bead of said plurality of beads is conjugated to the encoded polypeptide; assaying the encoded polypeptide to identify one or more properties of the encoded polypeptide; sequencing the nucleic acid molecule encoding the polypeptide to identify a sequence of the nucleic acid molecule encoding the polypeptide; and linking the one or more properties of each polypeptide to the sequence of the nucleic acid molecule encoding the polypeptide.

In some embodiments, the plurality of beads includes at least 1×10⁵ beads (e.g., at least 1×10⁶ beads, 1×10⁷ beads, 1×10⁸ beads, or 1×10⁹ beads, and values in between) where each bead is conjugated to a polypeptide (e.g., each polypeptide has a unique amino acid sequence).

In some embodiments, sequencing of the nucleic acid molecule and assaying the one or more functions or properties of each polypeptide are performed (e.g., sequentially, in any order) on the same machine, device, or instrument. In some embodiments, multiple assays are performed to determine two or more functions or properties of each polypeptide or multiple assays are performed to determine a single function or property of each polypeptide at varying condition. Multiple assays may be performed simultaneously or sequentially on the same machine, device, or instrument. For example, a single machine, device, or instrument may be used to sequence the nucleic acid molecule conjugated to each bead in order to identify the polypeptide conjugated to that bead; and to perform one or more assays to characterize each polypeptide (e. g., binding affinity, binding specificity, enzymatic activity, stability, e.g., at varying experimental conditions including, e.g., temperature and/or pH). In preferred embodiments, the sequencing and one or more assays produce fluorescence signatures that are measured by the single machine, device, or instrument.

In some embodiments, the encoded polypeptide is conjugated (e.g., covalently or non-covalently linked) directly to the bead. In other embodiments, the encoded polypeptide is conjugated (e.g., covalently or non-covalently linked) to the nucleic acid molecule, which is conjugated directly to the bead, thereby conjugating the polypeptide to the bead.

In some embodiments, the steps of conjugating each bead to a nucleic acid molecule, expressing the nucleic acid molecule to produce the polypeptide, and conjugating the polypeptide to the bead (e.g., directly or by conjugation to the nucleic acid) are performed in a first compartment (e.g., a first microemulsion droplet, tube, or microwell). In some embodiments, the method further includes amplifying each nucleic acid molecule within each compartment (e.g., within each microemulsion droplet), thereby producing a homogeneous population of a nucleic acid molecule on each bead. The amplified nucleic acids molecules may be conjugated to the bead within the first compartment (e.g., the first microemulsion droplet)

In some embodiments, expressing the nucleic acid molecule to produce the polypeptide; and

conjugating the polypeptide to the bead (e.g., directly or by conjugation to the nucleic acid) are performed in a second compartment (e.g., a second microemulsion droplet).

In some embodiments expressing the nucleic acid molecule to produce the polypeptide occurs in vitro in a cell free system.

In some embodiments, the nucleic acid is DNA, cDNA, or RNA. Where the nucleic acid is DNA or cDNA, expressing the nucleic acid refers to transcription of the DNA to RNA and translation of the RNA to produce the encoded polypeptide (e.g., in vitro transcription and translation (IVTT)). Where the nucleic acid is RNA, expression of the nucleic acid refers to translation of the RNA to produce the encoded polypeptide (e.g., in vitro translation (IVT)).

The disclosure provides methods for conjugating the polypeptide to the bead (e.g., via conjugation to the nucleic acid which is further conjugated to the bead). Such methods produce smaller, and/or more stable methods for linking a polypeptide and a nucleic acid to a bead. This allows assays to be performed at an increased range of conditions (e.g., temperature, pH, or salt concentration). Furthermore, a smaller assembly on the bead decreases nonspecific or off-target interactions with conjugation assembly components, thereby producing, a more accurate characterization of the plurality of polypeptides.

In another aspect, the disclosure provides a method of conjugating a polypeptide to a bead, the method including: in a first compartment (e.g., microemulsion droplet), conjugating a nucleic acid molecule encoding the polypeptide to a bead; and in a second compartment (e.g., microemulsion droplet), expressing the nucleic acid molecule to produce the polypeptide, and conjugating the polypeptide to the nucleic acid molecule, thereby conjugating the polypeptide to the bead.

In an aspect, the disclosure provides a method of conjugating a polypeptide to a bead, the method comprising: conjugating a nucleic acid molecule encoding the polypeptide to a bead in a first microemulsion droplet; and processing the nucleic acid molecule in a second microemulsion droplet, wherein processing comprises: expressing the nucleic acid molecule to produce the polypeptide; and conjugating the polypeptide to the nucleic acid molecule.

In some embodiments, conjugation of the polypeptide to the nucleic acid molecule is catalyzed by a linking enzyme. In some embodiments, the polypeptide is conjugated to the nucleic acid molecule by expressed protein ligation or by protein trans-splicing. In some embodiments, the polypeptide is conjugated to the nucleic acid molecule by formation of a leucine zipper;

In some embodiments, the bead or the nucleic acid molecule is conjugated to a capture moiety and the polypeptide includes a linkage tag, wherein the capture moiety and the linkage tag are conjugated, thereby conjugating the bead to the polypeptide or conjugating the nucleic acid molecule to the polypeptide.

In some embodiments, the conjugation of the capture moiety and the linkage tag is catalyzed by a linking enzyme. In some embodiments, the linking enzyme is encoded by a second nucleic acid. In some embodiments, the linking enzyme is simultaneously expressed with the polypeptide by addition of an encoding nucleic acid during IVTT or IVT (e.g., by addition of the nucleic acid encoding the linking enzyme during the second compartmentalization step, e.g., the second microemulsion step).

In some embodiments, the linking enzyme is an isolated enzyme (e.g., a purified, recombinant enzyme introduced into the second compartmentalization step, e.g., the second microemulsion droplet).

In some embodiments the linking enzyme is a sortase, a butelase, a trypsiligase, a peptiligase, a formylglycine generating enzyme, a transglutaminase, a tubulin tyrosine ligase, a phosphopantetheinyl transferase, a SpyLigase, or a SnoopLigase.

In some embodiments, the linking enzyme is sortase A. In other embodiments, where the linking enzyme is sortase A, one of the capture moiety or linkage tag includes a polypeptide which has a free N-terminal glycine residue. In another embodiment, the other of the capture moiety or linkage tag includes a polypeptide including amino acid sequence LPXTG (SEQ ID NO: 1), where X is any amino acid.

In some embodiments, the linking enzyme is butelase-1. In another embodiment, where the linking enzyme is butelase-1, one of the capture moiety or linkage tag includes a polypeptide including the amino acid sequence X₁X₂XX (SEQ ID NO: 2), where X₁ is any amino acid except P, D, or E; X₂ is I, L, V, or C; and X is any amino acid. In other embodiments, the other of the capture moiety or linkage tag includes a polypeptide including the amino acid sequence DHV or NHV.

In some embodiments, the linking enzyme is trypsiligase. In another embodiment, where the linking enzyme is trypsiligase, one of the capture moiety or linkage tag includes a polypeptide including amino acid sequence RHXX (SEQ ID NO: 3) where X is any amino acid. In another embodiment, the other of the capture moiety or linkage tag includes a polypeptide including the amino acid sequence YRH.

In some embodiments, the linking enzyme is omniligase. Where the linking enzyme is omniligase, the capture moiety may include carboxamido-methyl (OCam). In another embodiment, the linkage tag includes a polypeptide including a free N-terminal amino acid acting as an acyl-acceptor nucleophile.

In some embodiments, the linking enzyme is formylglycine generating enzyme. In other embodiments, where the linking enzyme is formylglycine, the capture moiety includes an aldehyde reactive group. For example, the linkage tag may include a polypeptide including the amino acid sequence CXPXR (SEQ ID NO: 4), where X is any amino acid.

In some embodiments, the linking enzyme is transglutaminase. Where the linking enzyme is transglutaminase, one of the capture moiety or linkage tag may include a polypeptide including a lysine residue or a free N-terminal amine group. In another embodiment, the other of the capture moiety or linkage tag includes a polypeptide including the amino acid sequence LLQGA (SEQ ID NO: 5).

In some embodiments, the linking enzyme is a tubulin tyrosine ligase. In other embodiments, where the linking enzyme is tubulin tyrosine ligase, one of the capture moiety or linkage tag includes a polypeptide including a free N-terminal tyrosine residue. For example, the other of the capture moiety or linkage tag may include a polypeptide including the C-terminal amino acid sequence VDSVEGEEEGEE (SEQ ID NO: 6).

In some embodiments, the linking enzyme is a tubulin phosphopantetheinyl transferase. In an embodiment where the linking enzyme is a tubulin phosphopantetheinyl transferase, the capture moiety may include coenzyme A (CoA). In another embodiment, the linkage tag includes a polypeptide including the amino acid sequence DSLEFIASKLA (SEQ ID NO: 7).

In some embodiments, the linking enzyme is SpyLigase. Where the linking enzyme is SpyLigase, one of the capture moiety or linkage tag may include a polypeptide including amino acid sequence ATHIKFSKRD (SEQ ID NO: 8). In other embodiments, the other of the capture moiety or linkage tag includes a polypeptide including the amino acid sequence AHIVMVDAYKPTK (SEQ ID NO: 9).

In some embodiments, the linking enzyme is SnoopLigase. In another embodiment, where the linking enzyme is SnoopLigase, one of the capture moiety or linkage tag includes a polypeptide including amino acid sequence DIPATYEFTDGKHYITNEPIPPK (SEQ ID NO: 10). In other embodiments, the other of the capture moiety or linkage tag includes a polypeptide including the amino acid sequence KLGSIEFIKVNK (SEQ ID NO: 11).

In some embodiments, the capture moiety includes double-stranded DNA and the linkage tag includes a polypeptide, in which the capture moiety and the linkage tag form a leucine zipper. In some embodiments, the capture moiety includes the nucleic acid sequence TGCAAGTCATCGG (SEQ ID NO: 12). In an embodiment where the capture moiety includes nucleic acid sequence TGCAAGTCATCGG (SEQ ID NO: 12), the linkage tag may include the amino acid sequence DPAALKRARNTEAARRSRARKGGC (SEQ ID NO: 13).

In some embodiments of any of the above, where the linkage tag or capture moiety includes a polypeptide sequence, the polypeptide sequence shares at least 70%, 75%, 80%, 85%, 90%, 95%, or 98% sequence identity with, or the sequence of, the exemplified polypeptide sequence.

In some embodiments, each bead is conjugated to 100 or more copies of the nucleic acid molecule (e.g., 150, 200, 250, 300, 350, 400, 500, 1000 or more copies).

In some embodiments, each bead is conjugated to 100 or more copies of the encoded polypeptide (e.g., 150, 200, 250, 300, 350, 400, 500, 1000 or more copies).

In some embodiments, the plurality of beads includes between 1×10⁶ and 1×10¹⁰ beads (e.g., between 2×10⁶ and 9×10⁹ beads, 4×10⁶ and 7×10⁹ beads, 6×10⁶ and 5×10⁹ beads, 8×10⁶ and 2×10⁹ beads, 1×10⁷ and 1×10¹⁰ beads, 1×10⁸, and 1×10¹⁰ beads, or 1×10⁹ and 1×10¹⁰ beads). In another embodiment, each bead is conjugated to a polypeptide having a unique amino acid sequence (e.g., each bead displays multiple copies of the unique polypeptide).

In some embodiments, the plurality of beads includes between 1×10⁶ and 1×10¹⁰ polypeptides having a unique amino acid sequence (e.g., between 2×10⁶ and 9×10⁹, 4×10⁶ and 7×10⁹ unique polypeptides, 6×10⁶ and 5×10⁹ unique polypeptides, 8×10⁶ and 2×10⁹ unique polypeptides, 1×10⁷ and 1×10¹⁰ unique polypeptides, 1×10⁸, and 1×10¹⁰ unique polypeptides, or 1×10⁹ and 1×10¹⁰ unique polypeptides). Each unique polypeptide may be represented multiple times in the library (e.g., either by multiple copies of the unique polypeptide being conjugated to a single or multiple beads).

Each polypeptide amino acid sequence may be represented on one or more beads with the plurality of beads. In some embodiments, the plurality of beads includes one or more, two or more, three or more, four or more, five or more, six or more, seven or more, eight or more, nine or more, or ten or more beads conjugated to one or more copies of the polypeptide having the unique amino acid sequence. In some embodiments, the plurality of beads includes between 1 and 15 beads (e.g., between 1 and 5, 1 and 10, 1 and 15, 2 and 5, 2 and 10, 2 and 15, 5 and 10, or 10 and 15 beads) conjugated to one or more copies of the polypeptide having the unique amino acid sequence.

In some embodiments, a function or property of each polypeptide is assayed at a high temperature (e.g., greater than or equal to 40° C., greater than or equal to 50° C., greater than or equal to 60° C., greater than or equal to 70° C., greater than or equal to 80° C., greater than or equal to 90° C., or greater than or equal to 100° C., such as between about 45° C. and about 100° C., between about 50° C. and about 90° C., between about 60° C. and about 80° C., or between about 65° C. and about 75° C.).

In some embodiments, the function or property of each polypeptide is assayed at a high pH (e.g., greater than or equal to pH 8.0, greater than or equal to pH 8.5, greater than or equal to pH 9.0, greater than or equal to pH 9.5, or greater than or equal to pH 10.0, such as between about pH 8.0 and about pH 10.0, between about pH 8.1 and about pH 9.9, or between about pH 8.2 and about pH 9.8).

In some embodiments, the function or property of each said polypeptide is assayed at a low pH (e.g., less than or equal to pH 6.0, less than or equal to pH 5.0, less than or equal to pH 4.0, or less than or equal to pH 3.0, such as between about pH 3.0 and about pH 6.0, or between about pH 3.1 and about pH 5.9, or between about pH 3.2 and about pH 5.8).

In some embodiments, the function or property of each polypeptide is assayed at a neutral pH (e.g., between about pH 6.0 and about pH 8.0, such as between about pH 7.0 and about pH 7.5).

In some embodiments, the one or more functions or properties of the polypeptide is a binding property, for example, quantification of binding to a molecule or a macromolecule (e.g., ligand binding, equilibrium binding, or kinetic binding, as described herein). In some embodiments, the function or property is enzymatic activity or specificity (e.g., enzyme activity or enzyme inhibition, as described herein). In some embodiments, the function or property is the level of protein expression (e.g., the expression level of a given gene). In some embodiments, the function or property of the polypeptide is stability (e.g., thermostability, e.g., as measured by thermal denaturation, chemical stability, e.g., as measured by chemical denaturation, or stability at varying pHs). In some embodiments, the function or property of the polypeptide is aggregation of the polypeptide.

In some embodiments, the method includes assaying multiple functions or properties of each polypeptide in the plurality of polypeptides (e.g., on a single machine, instrument, or device). For example, the method may include a determination of competitive binding to a target in the presence of a competitive molecule; measuring binding to multiple different targets; measuring equilibrium binding and binding kinetics; measuring binding and protein stability; or any combination thereof. The present methods may also include assaying multiple functions or properties of each polypeptide under varying conditions, e.g., binding under multiple pH conditions; binding under multiple temperature conditions; binding under multiple salt concentrations; and/or binding under multiple buffer conditions. The ability to perform multiple assays under varying conditions on a single instrument, where the instrument also performs a sequencing step (of a conjugated nucleic acid molecule) to identify the polypeptide being assayed, is a significant advantage of the compositions and methods of the present disclosure. Furthermore, multiple assays may be performed on the same library of polypeptides, thus improving the efficiency and speed relative to prior art methods.

In some embodiments, the plurality of polypeptides includes a library of antigens, antibodies, enzymes, substrates, or receptors. In some embodiments, the library of antigens includes viral protein epitopes for one or more viruses. In some embodiments, the plurality of polypeptides includes a library of enzymes (e.g., candidate enzymes) either derived from nature, implied from an organism's genomic data, or previously discovered through directed evolution. In some embodiments, the plurality of polypeptides includes a library of enzyme substrates for probing new or modified enzyme activity. In some embodiments, the plurality of polypeptides may encode partial or incomplete protein structures that interact with complementary protein fragments to form complete, functional proteins (e.g., protein-fragment complementation).

### Definitions

To facilitate the understanding of this invention, a number of terms are defined below. Terms defined herein have meanings as commonly understood by a person of ordinary skill in the areas relevant to the invention. Terms such as “a”, “an,” and “the” are not intended to refer to only a singular entity, but include the general class of which a specific example may be used for illustration. The terminology herein is used to describe specific embodiments of the invention, but their usage does not limit the invention, except as outlined in the claims.

As used herein, the term “about” refers to a value that is within 10% above or below the value being described.

As used herein, any values provided in a range of values include both the upper and lower bounds, and any values contained within the upper and lower bounds.

The terms “assay” or “assaying” as used herein refer to the measurement of a biological, and/or chemical, and/or physical property and/or function of a molecule. Examples of assays measurement of binding affinity, enzymatic activity, or thermostability of a protein, e.g., in a range of conditions such as temperature, pH, or salt concentrations.

The terms “amplification” or “amplify” or derivatives thereof, as used herein, mean one or more methods known in the art for copying a target or template nucleic acid, thereby increasing the number of copies of a selected nucleic acid sequence. Amplification may be exponential or linear. A “target nucleic acid” refers to a nucleic acid or a portion thereof that is to be amplified, detected, and/or sequenced. A target or template nucleic acid may be any nucleic acid, including DNA or RNA. The sequences amplified in this manner form an “amplified target nucleic acid,” “amplified region,” or “amplicon,” which are used interchangeably herein. Primers and/or probes can be readily designed to target a specific template nucleic acid sequence. Exemplary amplification approaches include but are not limited to polymerase chain reaction (PCR), ligase chain reaction (LCR), multiple displacement amplification (MDA), strand displacement amplification (SDA), rolling circle amplification (RCA), loop mediated isothermal amplification (LAMP), nucleic acid sequence based amplification (NASBA), helicase dependent amplification, recombinase polymerase amplification, nicking enzyme amplification reaction, and ramification amplification (RAM).

As used herein, a “bead” refers to a generally spherical or ellipsoid particle. The bead may be a solid or semi-solid particle. The bead may be composed of any one of various materials, including glass, quartz, silica, metal, ceramic, plastic, nylon, polyacrylamide, resin, hydrogel, and, composites thereof. The bead may be a gel bead (e.g., a hydrogel bead). The bead may be formed of a polymeric material. The bead may be magnetic or non-magnetic. Additionally, a substrate may be added to the surface of a bead to facilitate attachment of DNA templates (e.g., polyacrylamide matrix for immobilization of DNA templates carrying a terminal acrylamide group).

The term “bead aliquot” as used herein refers to a volume of beads comprising approximately 10,000-50,000 beads as measured using a flow cytometer. The actual volume of an aliquot can change depending on the concentration of the beads at the indicated step.

The term “capture moiety” as used herein refers to any molecule, natural, synthetic, or recombinantly-produced, or portion thereof, with the ability to bind to or otherwise associate with a target agent. Suitable capture moieties include, but are not limited to nucleic acids, antibodies, antigen-binding regions of antibodies, antigens, epitopes, cell receptors (e.g., cell surface receptors) and ligands thereof, such as peptide growth factors (see, e.g., Pigott and Power (1993), The Adhesion Molecule Facts Book (Academic Press New York); and Receptor Ligand Interactions: A Practical Approach, Rickwood and Hames (series editors) Hulme (ed.) (IRL Press at Oxford Press NY)). Similarly capture moieties may also include but are not limited to toxins, venoms, intracellular receptors (e.g., receptors which mediate the effects of various small ligands, including steroids, hormones, retinoids and vitamin D, peptides) and ligands thereof, drugs (e.g., opiates, steroids, etc.), lectins, sugars, oligosaccharides, other proteins, phospholipids, and structured nucleic acids such as aptamers and the like. Those of skill in the art readily will appreciate that molecular interactions other than those listed above are well described in the literature and may also serve as capture moiety/target agent interactions. In certain embodiments, capture moieties are associated with scaffolds, and in other embodiments capture moieties are conjugated to capture-associated oligos.

The term “cell free system” or “in vitro transcription/translation system” or “in vitro transcription/translation reaction mixture” or simply “reaction mixture” are synonymously used herein, and refer to a complex mixture of required components for carrying out transcription and/or translation in vitro, as recognized in the art. Such a reaction mixture may be a cell lysate such as an E. coli S30 extract, preferably from an E. coli cell lacking one or more release factors, e.g., Release Factor I (RF-I), Release Factor II (RF-II), and/or Release Factor III (RF-III), (Short, Biochemistry 1999, 38, pp: 8808-8819), or from a cell lacking a specific tRNA where the corresponding codon is to be used in the method of this invention as a stop codon. The reaction mixture may additionally include inhibitory components or constituents, that reduce the formation of unwanted by-products. Further the reaction mixture may include specific enzymes that actively remove one or more unwanted by-products. Further the reaction mixture may include specific enzymes that assist in ligation or improved folding or display of the polypeptide. Other such reaction mixtures may be artificially reconstituted from single components that may be purified from natural or recombinant sources.

As used herein, the term “clonal population” refers to a population of nucleic acids that is homogeneous with respect to a particular nucleotide sequence. The homogenous sequence can be at least 10 nucleotides long, or longer (e.g., at least 50, 100, 250, 500, 1000, 2000, or 4000 nucleotides long). A clonal population can be derived from a single target nucleic acid or template nucleic acid. Essentially all of the nucleic acid molecules in a clonal population have the same nucleotide sequence. It will be understood that a small number of mutations (e.g., due to PCR amplification artifacts) can occur in a clonal population without departing from clonality.

A “coding sequence” or a sequence which “encodes” a selected polypeptide is a nucleic acid molecule which is transcribed (in the case of DNA) and translated (in the case of mRNA) into a polypeptide. The boundaries of the coding sequence can be determined by a start codon at the 5′ (amino) terminus and a translation stop codon at the 3′ (carboxy) terminus. A coding sequence can include, but is not limited to, cDNA from viral, prokaryotic or eukaryotic mRNA, genomic DNA sequences from viral or prokaryotic DNA, and even synthetic DNA sequences. A transcription termination sequence may be located 3′ to the coding sequence.

The term “compartment” as used herein, refers the physical separation of one or more components from one or more other components. For example, compartmentalization may be used to perform a specific biological and/or chemical reaction, such as one or more of amplification of a nucleic acid molecule, conjugation of a nucleic molecule to a physical support (e.g., a bead), expression of a polypeptide encoded by a nucleic acid molecule (e.g., IVTT or IVT), or conjugation of a polypeptide to a physical support (e.g., by conjugation to the nucleic acid molecule). Exemplary compartments include, e.g., reaction tubes and microemulsion droplets,

As used herein, “conjugated” means attached or bound by covalent bonds, non-covalent bonds, and/or linked via Van der Waals forces, hydrogen bonds, and/or other intermolecular forces.

As used herein, the term “express” refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

The term “expressed protein ligation” or “EPL,” as used herein, refers to a protein semi-synthesis method that permits the in vitro ligation of a chemically synthesized C-terminal segment of a protein to a recombinant N-terminal segment fused through its C terminus to an intein protein splicing element. As used herein, the terms “function” and “property” refer to structural, regulatory, or biochemical activity of a naturally occurring and/or non-naturally occurring molecule including a protein or peptide, or fragment thereof. For example, a function of a fragment could include enzymatic activity (e.g., kinase, protease, phosphatase, glycosidase, acetylase, or transferase) or binding activity (e.g., binding DNA, RNA, protein, hormone, ligand, or antigen) of a functional protein domain.

The term “isolated enzyme”, as used herein refers to an externally purified enzyme that forms part of the reaction linking a polypeptide of interest to its encoding nucleic acid molecule. The isolated enzyme may be introduced into the reaction as a supplemental gene so that it is produced concurrently with the protein of interest or as a separate purified component.

As used herein, the term “linking enzyme” refers to an enzyme useful for the linkage reaction between a linkage tag and a capture moiety. Exemplary linking enzymes are described in detail herein.

The term “linkage tag”, as used herein, refers to a moiety (e.g., a polypeptide or small molecule) that interacts with a capture moiety. Where the capture moiety is bound to a first entity (e.g., a bead, a nucleic acid, or a polypeptide) and the linkage tag is bound to a second entity (e.g., a bead, a nucleic acid, or a polypeptide), interaction of the capture moiety and the linkage tag conjugates the first entity and the second entity. In preferred embodiments, interaction of the linkage tag and the capture moiety forms a covalent bond. In preferred embodiments, the linkage tag is a polypeptide (e.g. a short polypeptide of about 1-40, about 1-30, about 1-20, about 1-15, or about 1-10 amino acid residues). Covalent conjugation of a linkage tag to a capture moiety may be performed as escribed herein, for example, by conjugation by a linking enzyme.

The term “microemulsion” as used herein, refers to compositions including droplets in a medium, the droplets usually having diameters in the 100 nm to 10 μm range, that exist as single-phase liquid solutions that are thermodynamically stable.

The terms “nucleic acid” and “polynucleotide,” used interchangeably herein, refer to a polymeric form of nucleosides in any length. Typically, a polynucleotide is composed of nucleosides that are naturally found in DNA or RNA (e.g., adenosine, thymidine, guanosine, cytidine, uridine, deoxyadenosine, deoxythymidine, deoxyguanosine, and deoxycytidine) joined by phosphodiester bonds. The term encompasses molecules containing nucleosides or nucleoside analogs containing chemically or biologically modified bases, modified backbones, etc., whether or not found in naturally occurring nucleic acids, and such molecules may be preferred for certain applications. The term nucleic acid also encompasses natural nucleic acids modified during or after synthesis, conjugation, and/or sequencing. Where this application refers to a polynucleotide it is understood that both DNA (including cDNA), RNA, and in each case both single- and double-stranded forms (and complements of each single-stranded molecule) are provided. “Polynucleotide sequence” as used herein can refer to the polynucleotide material itself and/or to the sequence information (i.e., the succession of letters used as abbreviations for bases) that biochemically defines a specific nucleic acid. Various salts, mixed salts, and free acid forms of nucleic acid molecules are also included.

The terms “polypeptide,” “peptide,” “oligopeptide,” and “protein,” as used interchangeably herein, refer to any compound including naturally occurring or synthetic amino acid polymers or amino acid-like molecules including but not limited to compounds including amino and/or imino molecules. No particular size is implied by use of the term “peptide”, “oligopeptide”, “polypeptide”, or “protein.” The term, “protein,” as used herein refers to a full-length protein, portion of a protein, or a peptide. Included within the definition are, for example, polypeptides containing one or more analogs of an amino acid (including, for example, unnatural amino acids, etc.), polypeptides with substituted linkages, as well as other modifications known in the art, both naturally occurring and non-naturally occurring (e.g., synthetic). Thus, synthetic oligopeptides, dimers, multimers (e.g., tandem repeats, multiple antigenic peptide (MAP) forms, linearly-linked peptides), cyclized, branched molecules and the like, are included within the definition. The terms also include molecules including one or more peptoids (e.g., N-substituted glycine residues) and other synthetic amino acids or peptides (see, e.g., U.S. Pat. Nos. 5,831,005; 5,877,278; and U.S. Pat. No. 5,977,301; Nguyen et al. (2000) Chem. Biol. 7(7):463-473; and Simon et al. (1992) Proc. Natl. Acad. Sci. USA 89(20):9367-9371 for descriptions of peptoids). Non-limiting lengths of peptides suitable for use in the present invention includes peptides of 3 to 5 residues in length, 6 to 10 residues in length (or any integer therebetween), 11 to 20 residues in length (or any integer therebetween), 21 to 75 residues in length (or any integer therebetween), 75 to 100 (or any integer therebetween), or polypeptides of greater than 100 residues in length. Typically, polypeptides useful in this invention can have a maximum length suitable for the intended application. Further, polypeptides as described herein, for example synthetic polypeptides, may include additional molecules, such as labels or other chemical moieties. Such moieties may further enhance interaction of the peptides with a ligand and/or enhance detection of a polypeptide being displayed. Thus, reference to proteins, polypeptides, or peptides also includes derivatives of the amino acid sequences, including one or more non-naturally occurring amino acids.

A first polypeptide is derived from a second polypeptide if it is (i) encoded by a first polynucleotide derived from a second polynucleotide encoding the second polypeptide, or (ii) displays sequence identity to the second polypeptide as described herein. Sequence (or percent) identity can be determined as described below. Preferably, derivatives exhibit at least about 50% percent identity, more preferably at least about 80%, and even more preferably between about 85% and 99% (or any value therebetween) to the sequence from which they were derived. Such derivatives can include post-expression modifications of the polypeptide, for example, glycosylation, acetylation, phosphorylation, and the like. Amino acid derivatives can also include modifications to the native sequence, such as deletions, additions and substitutions (generally conservative in nature), so long as the polypeptide maintains the desired activity. These modifications may be deliberate, as through site-directed mutagenesis, or may be accidental, such as through mutations of hosts that produce the proteins or through errors during PCR amplification. Furthermore, modifications may be made that have one or more of the following effects: increasing efficiency of display, in vitro translation, function, or stability of the polypeptide.

As used herein, the term “protein trans-splicing” refers to protein splicing reactions that involve split intein systems. A split intein system refers to any intein system wherein a peptide bond break exists between the amino terminal and carboxy terminal amino acid sequences such that the N-terminal and C-terminal sequences become separate molecules which can re-associate, or reconstitute, into a functional trans-splicing element. The split intein system can be a naturally occurring split intein system, which encompasses any split intein systems that exist in natural organisms. The split intein system can also be an engineered split intein system, which encompasses any split intein systems that are generated by separating a non-split intein into an N-intein and a C-intein by any standard methods known in the art. As a non-limiting example, an engineered split intein system can be generated by breaking a naturally occurring non-split intein into appropriate N- and C-terminal sequences. Preferably, such engineered intein systems include only the amino acid sequences essential for trans-splicing reactions.

The term “sequencing” refers to any method for determining the nucleotide order of a nucleic acid (e.g., DNA), such as a target nucleic acid or an amplified target nucleic acid. Exemplary sequencing approaches include but are not limited to massively parallel sequencing (e.g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing)), long-read or single-molecule sequencing (e.g., Helicos™ sequencing, single-molecule real-time (SMRT™) sequencing, and nanopore sequencing) and Sanger sequencing. Massively parallel sequencing is also referred to in the art as next-generation or second-generation sequencing, and typically involves parallel sequencing of a large number (e.g., thousands, millions, or billions) of spatially-separated, clonally-amplified templates or single nucleic acid molecules. Short reads are often used in massively parallel sequencing. See, e.g., Metzker, Nature Reviews Genetics 11:31-36, 2010. Long-read sequencing and/or single-molecule sequencing are sometimes referred to as third-generation sequencing. Hybrid approaches (e.g., massively parallel and single molecule approaches or massively parallel and long-read approaches) can also be used. It is to be understood that some approaches may fall into more than one category, for example, some approaches may be considered both second-generation and third-generation approaches, and some sources refer to both second and third generation sequencing as “next-generation” sequencing.

### BRIEF DESCRIPTION OF THE DRAWINGS

FIG. 1 is a diagram illustrating an exemplary method of assaying a plurality of polypeptides. On a bead surface modified with a short DNA oligo (step 1), emulsion PCR is performed to display the polypeptide gene of interest (GOI) and relevant capture moiety (CM) which is covalently linked to the reverse primer (step 2). Emulsion in vitro transcription translation (IVTT) is performed to yield a linking enzyme and the target protein of interest (POI) containing a linkage tag (LT, step 3). During this step, the linking enzyme covalently fuses the CM to the LT resulting in covalent attachment of the POI. Emulsions are broken and the plurality of beads localized and physically addressed on the instrument (step 4). Beads are incubated with a fluorescent target of interest (TOI) to assay POI binding (step 5) via fluorescence measurements. The beads then undergo denaturation to leave behind only single-stranded DNA (ssDNA, step 6). The ssDNA undergoes sequencing by synthesis (step 7) to determine its identity which is fixed to the address determined in step 4. Upon sequencing, analysis yields biophysical data for the entire plurality of polypeptides encoded in the starting DNA library.

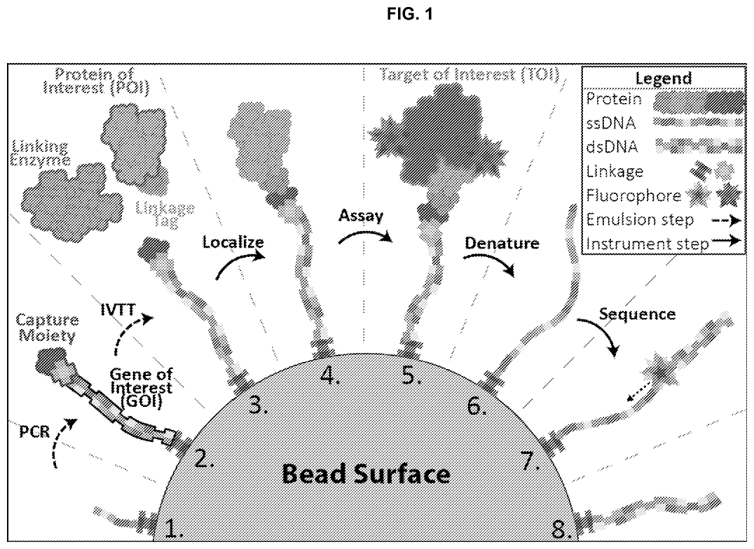

FIG. 2 is a schematic showing the structures and sequences of the biomolecules and/or peptide motifs on the DNA oligos (indicated by asterisks) and displayed on the proteins (indicated by arrowheads) used to covalently conjugate a protein of interest to its encoding DNA.

FIGS. 3A and 3B show histograms of events recorded via flow cytometry in the APC (660±20 nm) fluorescence channel upon excitation with a red laser (633 nm). (FIG. 3A) 10,000 events were collected from SA beads upon incubation with Alexa Fluor 647-labeled DNA. (FIG. 3B) Beads returned to baseline fluorescence levels upon stripping the Alexa Fluor 647-labelled anti-sense DNA strand using 20 mM sodium hydroxide.

FIGS. 4A and 4B are graphs showing the distribution of bead populations after fluorescent ddNTP incorporation (sequencing) in the 610±20 nm fluorescence channel upon excitation with a blue laser (488 nm) (FIG. 4A). Distribution of bead populations after sequencing in the 660±20 nm fluorescence channel upon excitation with a red laser (633 nm) (FIG. 4B).

FIGS. 5A-C show exemplary flow cytometry results. FIG. 5A is a schematic summary of an exemplary flow cytometry analysis. A bead displaying double-stranded DNA, its encoded polypeptide, and any bound fluorescent anti-FLAG M2 antibody was directed through the flow cytometer and excited by three consecutive lasers (blue, red, and violet). The signals produced upon blue laser excitation yield information regarding the amount of binding to the M2 antibody (assay, FITC channel) and the amount of fluorescent ddUTP incorporation (U, PE channel). The signal produced by red excitation yields information on the amount of fluorescent ddCTP or ddGTP (C/G, APC channel) incorporation. The signal produced upon violet laser excitation yields information on the amount of fluorescent ddATP (A, AmCyan channel) incorporation.

FIG. 5B is a plot showing the fluorescent signal of each bead in the relevant channels (APC, PE, AmCyan channels). The fluorescent signal in each channel was analyzed and the beads were assigned a base call which identifies the oligonucleotide being monoclonally displayed on the bead. Because of heterogenous signal generation, some beads do not yield sufficient fluorescence and their displayed oligonucleotide is undetermined. FIG. 5C is a set of graphs showing the fluorescent signal in the assay channel (FITC channel). The fluorescent signal was aggregated for each oligonucleotide population and the mean values were fit to obtain an accurate measurement of binding affinity (colored lines). Overlayed violin plots show the geometric mean (white circle), bars (thick lines) that extend from the first (25%) to the third (75%) quartile, and whiskers (thin lines) that extend to 1.5 times the interquartile range.

### DETAILED DESCRIPTION

The disclosure provides compositions and methods for assaying the function or properties of a plurality of polypeptides. In particular, the disclosure provides methods for high-throughput characterization of a large population(s) of polypeptides. Each polypeptide is displayed on a solid surface, such as a bead, where the solid surface also displays a nucleic acid that encodes the polypeptide. For example, each polypeptide may be covalently linked to a nucleic acid that encodes the polypeptide. In preferred embodiments, the polypeptide and nucleic acid are assayed in parallel, and with the same instrument. This enables characterization of large libraries of polypeptides. Multiple assays may be performed, one after another or simultaneously, on the same library of polypeptides without the need for selection, thus allowing each member to be characterized across multiple parameters in a less-costly and time intensive manner as compared to prior art methods.

### Methods for High Throughput Polypeptide Assays on Beads

Described herein are methods for high-throughput protein assays performed directly on beads. The high-throughput protein assay methods described herein include, in some embodiments, 1) generating a plurality of beads that each display a unique clonal population of protein encoding-DNA; 2) transcribing and translating the DNA displayed on each bead to generate a unique clonal population of protein variants corresponding to the clonal DNA population of each bead; 3) chemically linking the clonal protein molecules to the DNA molecules displayed on the beads to generate bead-DNA-protein conjugates; 4) characterizing in a common machine, and/or instrument, and/or device a plurality of physicochemical properties, and/or biochemical functions of the proteins of the bead-DNA-protein conjugates; 5) reading the sequences of the DNA molecules of the bead-DNA-protein conjugates to identify the DNA and thus protein sequence of the bead-DNA-protein conjugates; and 6) performing all steps with automation and/or with minimal user intervention. The successful implementation of the methods yields a high-throughput approach to protein assays eliminating the requirement for multiple rounds of conventional directed evolution. A more detailed overview of the steps and the uses of the methods is provided below.

### Displaying Polynucleotides on Beads

Methods for displaying clonal populations of polynucleotides on the surface of a plurality of beads are described. In some embodiments, an aqueous solution containing a library of nucleic acids, preferably DNA or cDNA (e.g., of at least 1×10⁵ variants, at least 1×10⁶ variants, at least 1×10⁷ variants, at least 1×10⁸ variants, at least 1×10⁹ variants, or at least 1×10⁶ variants, such as 1×10⁵ to 1×10¹⁰ variants, 5×10⁵ to 5×10⁸ variants, 1×10⁶ to 1×10⁸ variants, 5×10⁶ to 5×10⁷ variants, 1×10⁷ to 4×10⁷ variants, or 2×10⁷ to 3×10⁷ variants), surface-functionalized beads (e. g., beads with chemical groups added to the surface of each bead to facilitate attachment of the nucleic acid templates), and reagents for linking the nucleic acid to the surface of the functionalized beads, are combined to generate a mixture. The mixture is preferably in an aqueous medium. In some embodiments, nucleic acid variants will have a terminal reactive group that facilitates the immobilization of the nucleic acid variants to the surface functionalized beads. For example, each bead can be functionalized with a polyacrylamide matrix on the surface for immobilization of DNA templates carrying a terminal acrylamide group.

In some embodiments, nucleic acid variants will have a terminal small molecule moiety that facilitates immobilization to surface-functionalized beads. For example, each bead can be functionalized with streptavidin for immobilization of DNA templates containing a terminal biotin moiety. In some embodiments, each bead may be functionalized with carboxylic acid functional groups for covalent immobilization of DNA templates containing a terminal amine group. In some embodiments, DNA templates may be fully or partially synthesized on the bead surface via phosphoramidite chemistry as in, e.g., Diamante et al (2013) Protein Engineering Design and Selection 26 (10): 713-724, Sepp et al (2002) FEBS Letters 532 (2002): 455-458, and Griffiths and Tawfik (2003) EMBOJ 22(1): 24-35, herein incorporated by reference in their entireties. The mixture may be emulsified, e.g., in a first microemulsion, to create a large number (e. g., more than 1×10⁵, 1×10⁶, 1×10⁷, 1×10⁸, 1×10⁹, or 1×10¹⁰, such as 1×10⁵-1×10¹²) of water-in-oil droplets. The components of the mixture can be tuned, as described herein, to ensure that each droplet contains on average one bead and one or fewer nucleic acid template copies.

In some embodiments, the beads can be composed of any one of various materials, including glass, quartz, silica, metal, ceramic, plastic, nylon, polyacrylamide, resin, hydrogel, and, composites thereof. The bead may be a gel bead (e.g., a hydrogel bead). The bead may be formed of a polymeric material. The bead may be magnetic or non-magnetic. In particular embodiments, the beads are substantially homogeneous in size (plus/minus 5% variance) and contain sufficient functional handles to display, e.g., about 10³-10⁶ DNA molecules per bead.

In some embodiments, the nucleic acid in each droplet is amplified directly on the surface of the bead via extension of immobilized DNA oligos. In some embodiments, the nucleic acid may be separately amplified in a droplet containing no bead and then fused in a microfluidic channel with a separate droplet containing a bead. In some embodiments, upon generation of the emulsion droplets, the nucleic acid in each droplet is amplified via polymerase chain reaction to create a clonal population of each nucleic acid variant. Physical immobilization of the amplified nucleic acid in each microemulsion droplet can be achieved, e.g., via ligation or extension of immobilized DNA oligos to generate nucleic acid-coated beads (e.g., DNA-coated beads).

### Displaying Polypeptides on Beads

Methods for displaying polypeptides on the surface of a plurality of beads are described herein. Starting with nucleic acid-coated beads (e.g., DNA-coated beads), prepared using the methods for displaying polynucleotides on beads, the encoded polypeptide can be expressed and conjugated to the bead (e.g., via conjugation to the nucleic acid which is conjugated to the bead). Conjugation of the polypeptide to the bead (e.g., directly or via attachment to the nucleic acid) may be performed in a second microemulsion step.

For example, DNA-coated beads are emulsified in a second microemulsion, along with a mixture that includes reagents for cell-free in vitro transcription and translation (IVTT) methods resulting in the transcription and translation of the DNA on the beads and the production of the encoded polypeptide and/or protein. In some embodiments, the second microemulsion contains reagents for IVTT as well as a catalytic enzyme or solution-phase DNA which codes for a catalytic enzyme and catalyzes the attachment of the polypeptide to the capture moiety on the nucleic acid. The components of the mixture can be tuned, as described herein, to ensure on average one DNA-coated bead and sufficient IVTT reagents.

Protein expression may be carried out using an in vitro cell-free expression system. Translation can be performed in vitro using a crude lysate from any organism that provides all the components needed for translation, including, enzymes, tRNA and accessory factors (excluding release factors), amino acids and an energy supply (e.g., GTP). Cell-free expression systems derived from Escherichia coli, wheat germ, and rabbit reticulocytes are commonly used. E. coli-based systems provide higher yields, but eukaryotic-based systems are preferable for producing post-translationally modified proteins. Alternatively, artificial reconstituted cell-free systems may be used for protein production. For optimal protein production, the codon usage in the ORF of the DNA template may be optimized for expression in the particular cell-free expression system chosen for protein translation. In addition, labels or tags can be added to proteins to facilitate high-throughput screening. See, e.g., Katzen et al. (2005) Trends Biotechnol. 23:150-156; Jermutus et al. (1998) Curr. Opin. Biotechnol. 9:534-548; Nakano et al. (1998) Biotechnol. Adv. 16:367-384; Spirin (2002) Cell-Free Translation Systems, Springer; Spirin and Swartz (2007) Cell-free Protein Synthesis, Wiley-VCH; Kudlicki (2002) Cell-Free Protein Expression, Landes Bioscience; herein incorporated by reference in their entireties. In some embodiments the cell-free expression system uses a prokaryotic IVTT mix reconstituted from purified components (e.g., PURExpress). In some embodiments the IVTT includes an E. coli lysate-based system (e.g., S30) to facilitate increased scale (e.g., 10⁹ to 10¹⁰ beads). In some embodiments in vitro cell expression is performed using a eukaryotic system (e.g., wheat germ, rabbit reticulocyte, HeLa cell lysate-based,) in order to achieve proper folding or post-translational modification (PTM) of the proteins to be displayed. In some embodiments, the polynucleotides expressed using IVTT methods include non-natural amino acids.

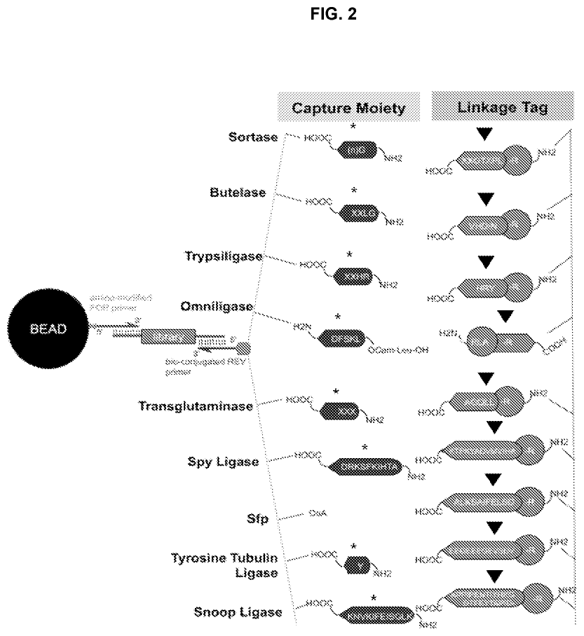

In other embodiments, the plurality of polypeptides can be linked to the DNA-bead conjugates to produce protein-DNA-bead conjugates. In some embodiments, linking of the protein to the DNA-coated bead is achieved using a three-part enzymatic linkage system. In some embodiments, the three-part enzymatic linkage system is composed of 1) a linking enzyme; 2) a capture moiety (e.g., a small molecule or peptide capture moiety) of the DNA on the DNA-coated beads; and 3) a linkage tag (e.g., a peptide linkage tag) of the protein (see, e.g., FIG. 2). Use of a three-part enzymatic linkage system may require a modification to the sequence of a polynucleotide encoding the protein to include the polynucleotide sequence encoding a capture moiety. In parallel, inclusion of a linkage tag moiety may be achieved by performing a modification to the sequence encoding the protein.

The disclosure also provides methods for conjugating polypeptides to beads (e.g., via conjugation to a nucleic acid which is further conjugated to a bead). Such methods produce smaller and/or more stable methods for linking a polypeptide and a nucleic acid to a bead. This allows assays to be performed at an increased range of conditions (e.g., temperature, pH, or salt concentration). Furthermore, a smaller assembly on the bead decreases off-target effects allowing for a more accurate characterization of the plurality of polypeptides.

In some embodiments, the method for conjugating a polypeptide to a bead (e.g., via conjugation to a nucleic acid which is further conjugated to a bead) includes: in a first microemulsion droplet, conjugating a nucleic acid molecule encoding the polypeptide to a bead; and in a second microemulsion droplet, expressing the nucleic acid molecule to produce the polypeptide, and concurrently conjugating the polypeptide to the nucleic acid molecule, thereby conjugating the polypeptide to the bead.

In other embodiments, conjugation of the polypeptide to the nucleic acid displayed on the bead is catalyzed by a linking enzyme. For example, the linking enzyme may be selected from a sortase, a butelase, a trypsiligase, a peptiligase, a formylglycine generating enzyme, a transglutaminase, a tubulin tyrosine ligase, a phosphopantetheinyl transferase, a SpyLigase, or a SnoopLigase.

Enzymatic linkage of a protein to a DNA molecule displayed on beads may be accomplished using Sortase A as the linking enzyme. In this embodiment, one of the capture moiety or linkage tag can include a polypeptide which has a free N-terminal glycine residue and the other of the capture moiety or linkage tag can include a polypeptide which has an amino acid sequence LPXTG (SEQ ID NO: 1), where X is any amino acid (see, e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using Butelase-1 as the linking enzyme. In this embodiment, one of the capture moiety or linkage tag can include a polypeptide including the amino acid sequence X₁X₂XX (SEQ ID NO: 2), where X₁ is any amino acid except P, D, or E; X₂ is I, L, V, or C; X is any amino acid, and the other of the capture moiety or linkage tag can include a polypeptide including the amino acid sequence DHV or NHV (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using Trypsiligase as the linking enzyme. In this embodiment, one of the capture moiety or linkage tag can include a polypeptide including amino acid sequence RHXX (SEQ ID NO: 3), where X is any amino acid, and the other of the capture moiety or linkage tag can include a polypeptide including the amino acid sequence YRH (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using a Subtilisin-derived enzyme (e. g., Omniligase) as the linking enzyme. In this embodiment, the capture moiety can include carboxamido-methyl (OCam) and the linkage tag can include a polypeptide including a free N-terminal amino acid acting as an acyl-acceptor nucleophile (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using a Formylglycine generating enzyme (FGE) as the linking enzyme. In this embodiment, the capture moiety can include an aldehyde reactive group and the linkage tag can include a polypeptide including the amino acid sequence CXPXR (SEQ ID NO: 4), where X is any amino acid (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using transglutaminase as the linking enzyme. In this embodiment, one of the capture moiety or linkage tag can include a polypeptide including a lysine residue or a free N-terminal amine group and the other of the capture moiety or linkage tag can include a polypeptide including the amino acid sequence LLQGA (SEQ ID NO: 5) (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using tubulin tyrosine ligase as the linking enzyme. In this embodiment, one of the capture moiety or linkage tag can include a polypeptide including a free N-terminal tyrosine residue and the other of the capture moiety or linkage tag can include a polypeptide including the C-terminal amino acid sequence VDSVEGEEEGEE (SEQ ID NO: 6) (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using tubulin phosphopantetheinyl transferase as the linking enzyme. In this embodiment, the capture moiety can include coenzyme A (CoA) and the linkage tag can include polypeptide including the amino acid sequence DSLEFIASKLA (SEQ ID NO: 7) (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using SpyLigase as the linking enzyme. In this embodiment, one of the capture moiety or linkage tag can include a polypeptide including amino acid sequence ATHIKFSKRD (SEQ ID NOL 8) and the other of the capture moiety or linkage tag can include a polypeptide including the amino acid sequence AHIVMVDAYKPTK (SEQ ID NO: 9) (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entireties).

Enzymatic linkage of a protein to a DNA molecule displayed on beads may be accomplished using SnoopLigase as the linking enzyme. In this embodiment, one of the capture moiety or linkage tag can include a polypeptide including amino acid sequence DIPATYEFTDGKHYITNEPIPPK (SEQ ID NO: 10) and the other of the capture moiety or linkage tag can include a polypeptide including the amino acid sequence KLGSIEFIKVNK (SEQ ID NO: 11) (see e.g., Schmidt et al (2017) Current Opinion in Chemical Biology 38: 1-7, Falck and Muller (2018) Antibodies 7(1): 4 and Massa and Devoogdt (2019) Bioconjugation: Methods and Protocols, herein incorporated by reference in their entirety).

In an embodiment, the capture moiety includes double-stranded DNA and the linkage tag includes a polypeptide, in which the capture moiety and the linkage tag form a leucine zipper. In another embodiment, the capture moiety includes the nucleic acid sequence TGCAAGTCATCGG (SEQ ID NO: 12) and the linkage tag includes the amino acid sequence DPAALKRARNTEAARRSRARKGGC (SEQ ID NO: 13) (see e.g., Stanojevic and Verdine (1995) Nat Struct Biol 2(6): 450-7, herein incorporated by reference in its entirety.

In some embodiments the linking enzyme is introduced into the mixture of the second microemulsion as a purified component. In some embodiments the linking enzyme is introduced into the second microemulsion in the form of a supplemental gene that is expressed concurrently with the protein variant library. Linking of the DNA on the DNA-coated beads to the linkage tag of the protein is performed to achieve a protein density of 10³ to 10⁶ molecules per μm² of bead surface area.

In other embodiments, the protein-DNA-bead conjugates display antigens, antibodies, enzymes, substrates or, receptors. In some embodiments the library of antigens displayed on the protein-DNA-bead conjugates includes protein epitopes for one or more pathogenic agents or cancers (e.g., 1-10 epitope variants, 1-9 epitope variants, 1-8 epitope variants, 1-7 epitope variants, 1-6 epitope variants, 1-5 epitope variants, 1-4 epitope variants, 1-3 epitope variants, 1-2 epitope variants, 1 epitope variant, 2 epitope variants, 3 epitope variants, 4 epitope variants, 5 epitope variants, 6 epitope variants, 7 epitope variants, 8 epitope variants, 9 epitope variants, or 10 epitope variants).

In some embodiments, the protein-DNA-bead conjugates display proteins associated with cancer. For example, the conjugates may display proteins associated with a cancer selected from acute lymphoblastic leukemia, acute myeloid leukemia, adrenocortical carcinoma, an AIDS-related cancer, an AIDS-related lymphoma, anal cancer, appendix cancer, an astrocytoma, basal cell carcinoma, bile duct cancer, bladder cancer, bone cancers, brain tumors, such as cerebellar astrocytoma, cerebral astrocytoma/malignant glioma, ependymoma, medulloblastoma, supratentorial primitive neuroectodermal tumors, visual pathway and hypothalamic glioma, breast cancer, a bronchial adenoma, Burkitt lymphoma, carcinoma of unknown primary origin, central nervous system lymphoma, cerebellar astrocytoma, cervical cancer, a childhood cancer, chronic lymphocytic leukemia, chronic myelogenous leukemia, a chronic myeloproliferative disorder, colon cancer, cutaneous T-cell lymphoma, desmoplastic small round cell tumor, endometrial cancer, ependymoma, esophageal cancer, Ewing's sarcoma, a germ cell tumor, gallbladder cancer, gastric cancer, gastrointestinal carcinoid tumor, gastrointestinal stromal tumor, a glioma, hairy cell leukemia, head and neck cancer, heart cancer, hepatocellular (liver) cancer, Hodgkin lymphoma, Hypopharyngeal cancer, intraocular melanoma, islet cell carcinoma, Kaposi sarcoma, kidney cancer, laryngeal cancer, lip and oral cavity cancer, liposarcoma, liver cancer, a lung cancer, such as non-small cell and small cell lung cancer, a lymphoma, a leukemia, macro globulinemia, malignant fibrous histiocytoma of bone/osteosarcoma, medulloblastoma, melanomas, mesothelioma, metastatic squamous neck cancer with occult primary, mouth cancer, multiple endocrine neoplasia syndrome, myelodysplasia syndromes, myeloid leukemia, nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer, ovarian germ cell tumor, pancreatic cancer, pancreatic cancer islet cell, paranasal sinus and nasal cavity cancer, parathyroid cancer, penile cancer, pharyngeal cancer, pheochromocytoma, pineal astrocytoma, pineal germinoma, pituitary adenoma, pleuropulmonary blastoma, plasma cell neoplasia, primary central nervous system lymphoma, prostate cancer, rectal cancer, renal cell carcinoma, renal pelvis and ureter transitional cell cancer, retinoblastoma, rhabdomyosarcoma, salivary gland cancer, sarcomas, a skin cancer, skin carcinoma merkel cell, small intestine cancer, soft tissue sarcoma, squamous cell carcinoma, stomach cancer, T-cell lymphoma, throat cancer, thymoma, thymic carcinoma, thyroid cancer, trophoblastic tumor (gestational), cancers of unknown primary site, urethral cancer, uterine sarcoma, vaginal cancer, vulvar cancer, Waldenstrom macro globulinemia, and Wilms tumor.

In some embodiments, the protein-DNA-bead conjugates display proteins associated with an infectious agent (e.g., viral proteins, bacterial proteins, fungal proteins, or parasitic proteins). For example, the conjugates may display proteins associated with a virus selected from COVID-19, HIV, Dengue, West Nile Virus (WNV), Syphilis, Hepatitis B Virus (HBV), Normal Blood, Valley Fever, and Hepatitis C Virus.

In some embodiments, the protein-DNA-bead conjugates display proteins associated with an inflammatory and/or autoimmune disease. In some embodiments, the inflammatory or autoimmune disease is selected from HIV, rheumatoid arthritis, diabetes mellitus type 1, systemic lupus erythematosus, scleroderma, multiple sclerosis, severe combined immunodeficiency (SCID), DiGeorge syndrome, ataxia-telangiectasia, seasonal allergies, perennial allergies, food allergies, anaphylaxis, mastocytosis, allergic rhinitis, atopic dermatitis, Parkinson's disease, Alzheimer's disease, hypersplenism, leukocyte adhesion deficiency, X-linked lymphoproliferative disease, X-linked agammaglobulinemia, selective immunoglobulin A deficiency, hyper IgM syndrome, autoimmune lymphoproliferative syndrome, Wiskott-Aldrich syndrome, chronic granulomatous disease, common variable immunodeficiency (CVID), hyperimmunoglobulin E syndrome, Hashimoto's thyroiditis, and/or a breakdown in cellular signaling processes.

### Microemulsion Droplets

Methods for producing microemulsion droplets for the purpose of chemical and biochemical reactions are known to those of skill in the art. In general, microemulsion droplets contain an aqueous phase suspended in an oil phase (e.g. a water-in-oil emulsion). In an embodiment, the oil phase is comprised of 95% mineral oil, 4.5% Span-80, 0.45% Tween-80, and 0.05% Triton X-100. In some embodiments, the microemulsions are formed via direct mixing and/or vortexing of aqueous and oil phases. In some embodiments, the microemulsions are formed via a piezoelectric pump extruding the aqueous phase in a microfluidic channel containing oil phase. In some embodiments, the microemulsions are formed via mechanical mixing of aqueous and oil phases using a dispersing instrument or homogenizer. In an embodiment, each emulsion droplet contains on average a single primer-coated bead, one template DNA molecule, and a plurality of PCR primer molecules. Temperature cycling can be used to produce clonal DNA amplified from the template on the beads.

### High-Throughput Characterization of Protein Properties and Functions

Methods for high-throughput assays of large pluralities of protein variants (e. g., at least 1×10⁵ variants, at least 1×10⁶ variants, 1×10⁷ variants, 1×10⁸ variants, or 1×10⁹ variants, such as between 1×10⁵ and 1×10¹⁰ variants, between 1×10⁶ and 1×10¹⁰ variants, or between 10×10⁷ and 1×10¹⁰ variants) on one automated instrument are described herein.

In particular embodiments, after protein generation and display in the second microemulsion, the emulsion can be broken, leaving the population of beads displaying many copies of a protein and many clonal copies of the DNA encoding the protein. Then, the beads can be introduced into an instrument that is configured to sequence the DNA of each bead and also analyze the properties and/or function of the displayed proteins in a high-throughput manner. In an embodiment, the beads can be immobilized onto a solid surface (e.g., collected into nanowells). The immobilized library of polypeptides can then be presented with various reagents (e.g., target drugs, epitopes, paratopes, or antigens) that can be flowed over the beads, the function and/or property of the polypeptides can be assayed via a fluorescence signal that is detected (e.g., fluorescence imaging) and quantified. In several embodiments, the reagents are then washed out and the process can be repeated (e.g., 2 times, 3 times, 4 times, 5 times, 6 times, 7 times, 8 times, 9 times, or 10 times). In some embodiments, a single assay run can include a first step of measuring equilibrium binding to a first target (target “A”), a second step of measuring binding kinetics to target A, a third step of measuring the equilibrium binding to a second target (target “B”), a fourth step of measuring the binding kinetics to target B, followed by a fifth step of measuring protein stability (e.g., denaturation) in a variety of environmental conditions (e.g., temperature, pH, and/or tonicity). In some cases, the order of assays can be selected to ensure that any resulting changes to the polypeptide (e.g., irreversible changes to the polypeptide, such as, e.g., denaturation) will not affect the readout. In some embodiments, a regeneration step can be performed after each assay to prepare the beads for subsequent assays. Regeneration steps can be configured to incubate the beads in a low pH solution (e.g., pH=4.5) to cause any bound molecules to dissociate, followed by, e.g., a washing step, and step that returns the beads to a state (e.g., neutral pH) that can be used in the next assay. Regeneration via low pH presents an advantage of the methods of the present disclosure and an advancement over the prior art methods due to the nature of the covalent bonding between the constituents of the protein-DNA-bead conjugates. Regeneration with low pH in methods previously established in the field is not possible, given that such exposure to low pH results in the irreversible disruption of protein-DNA conjugates that limits or precludes the possibility of performing subsequent assays.

In some embodiments, the methods described herein can be configured to perform a wide variety of assays to characterize a polypeptide (e.g., equilibrium binding assay (K_{d}), kinetic binding assay (association, kₒₙ), kinetic binding assay (dissociation, k_{off}), limit of detection assay (LoD), thermal denaturation (equilibrium unfolding, Tm), and/or chemical denaturation (equilibrium unfolding, C_{1/2})). In some embodiments, the kinetic stability of a polypeptide is measured by a first step of adding a reagent (e.g., a target drug, antigen, epitope, paratope, or orthogonal antibody) to a displayed protein and a second step of increasing the temperature and/or increasing the concentration of a denaturant until a binding signal (e.g., fluorescence signal) disappears.

In some embodiments the protein variants of the protein-DNA-bead conjugates are evaluated for properties including, e.g., thermal stability and pH stability.

In some embodiments, the thermal stability of protein variants of the protein-DNA-bead conjugates is performed by characterizing the denaturation of the protein variants in response to elevated temperatures (e. g., greater than 45° C., between 45° C.-100° C., between 55° C.-90° C., between 65° C.-80° C., between 45° C.-90° C., between 55° C.-80° C., between 65° C.-70° C., between 45° C.-55° C. between 55° C.-65° C., between 65° C.-75° C., between 75° C.-85° C., between 85° C.-95° C. between 95° C.-100° C., between 40° C.-45° C., between 46° C.-50° C., between 50° C.-55° C., between 55° C.-60° C., between 60° C.-65° C., between 65° C.-70° C., between 70° C.-75° C., between 75° C.-80° C., between 80° C.-85° C., between 85° C.-90° C., between 90° C.-95° C., between 95° C.-100° C., or at or above 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.). In some embodiments, the denaturation of the protein variants in response to elevated temperatures is evaluated using fluorescent detection of denatured proteins (e. g., FACS sorting).

In some embodiments, the pH stability of protein variants of the protein-DNA-bead conjugates is performed by characterizing the denaturation of the protein variants in response to a low pH (e. g., below pH 6.0, such as between pH 3.0-6.0, or between pH 4.0-5.0, or between pH 3.0-3.5, or between pH 3.5-4.0, or between pH 4.0-4.5, or between pH 4.5-5.0, or between pH 5.0-5.5, or between pH 5.5-6.0, or pH 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0). In some embodiments, the denaturation of the protein variants in response to low pH is evaluated using fluorescent detection of denatured proteins (e. g., FACS sorting).

In some embodiments, the pH stability of protein variants of the protein-DNA-bead conjugates is performed by characterizing the denaturation of the protein variants in response to high pH (e. g., above pH 8.0, such as between pH 8.0-10.0, or between pH 8.0-8.5, or between pH 8.5-9.0, between pH 9.0-9.5, or between pH 9.5-10.0, or pH 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10.0). In some embodiments, the denaturation of the protein variants in response to high pH is evaluated using fluorescent detection of denatured proteins (e. g., FACS sorting).

In some embodiments, biological activity (e. g., binding affinity, binding specificity, and/or enzymatic activity) of a large plurality of protein variants, displayed on protein-DNA-bead conjugates, is characterized on one automated instrument. In an embodiment, the binding affinity of protein variants is determined using fluorescent detection of binding between protein variants and fluorescently-labeled target molecules (e. g., agonists, antagonists, competitive inhibitors and or, allosteric inhibitors). In another embodiment, the binding specificity of protein variants is determined using fluorescent detection of binding between protein variants and fluorescently-labeled target molecules (e. g., agonists, antagonists, competitive inhibitors and/or, allosteric inhibitors). In some embodiments the binding affinity and binding specificity are determined for a large plurality of protein variants sequentially in any order on one automated instrument. In some embodiments, the enzymatic activity of a large plurality of protein variants, displayed on protein-DNA-bead conjugates, is characterized on one automated instrument. In an embodiment, the enzymatic activity is determined using fluorescent detection of the increase of reaction product(s) and/or using fluorescent detection of the decrease of reactant reagent(s).

The protein-DNA-bead conjugates can be used to interrogate the interaction of a biologic molecule (e.g., an antibody, a paratope, an antigen, an enzyme, a substrate, or a receptor) and a drug (e.g., an antiviral drug, Abciximab, Adalimumab, Alefacept, Alemtuzumab, Basiliximab, Belimumab, Bezlotoxumab, Canakinumab, Certolizumab pegol, Cetuximab, Daclizumab, Denosumab, Efalizumab, Golimumab, Inflectra, Ipilimumab, Ixekizumab, Natalizumab, Nivolumab, Olaratumab, Omalizumab, Palivizumab, Panitumumab, Pembrolizumab, Rituximab, Tocilizumab, Trastuzumab, Secukinumab, Ustekinumab, or Cabliv).

In other embodiments, the protein-DNA-bead conjugates can be used in a diagnostic and/or a companion diagnostic process. In some embodiments the protein-DNA-bead conjugates may display a variety of patient-specific drug targets to test effectiveness of a drug that is bound to the protein-DNA-bead conjugates as part of a companion diagnostic for the drug. In some embodiments the protein-DNA-bead conjugates can be used to display patient-specific cancer epitope variants (e.g., neoantigens) in order to test drug effectiveness against the patient's cancer-specific variants. In some embodiments, the protein-DNA-bead conjugates can be used to display patient- or population-specific epitopes associated with an infectious agent to characterize bacterial or viral drug resistance and drug effectiveness.

In some embodiments the protein-DNA-bead conjugates can be used to display a biomarker or other diagnostic epitope, then incubated with a patient's serum, in which the patient's antibodies in the serum bind to the protein-DNA-bead conjugates and are detected with a secondary anti-human antibody to assay a patient's antibody responses as a diagnostic. In some embodiments, the protein-DNA-bead conjugates can be configured to display allergen epitopes in order to diagnose and characterize a subject's allergic response. In some embodiments, the protein-DNA-bead conjugates can be configured to display a wide variety and of epitopes from a broad group of infectious agents to test the serum of a patient and diagnose active infections and also to characterize immune protection (e.g., immunization).

In some embodiments, the function or property of the polypeptide is binding to a target (e.g., ligand binding, equilibrium binding, or kinetic binding as described herein). In some embodiments, the function or property is enzymatic activity or specificity (e.g., enzyme activity or enzyme inhibition as described herein). In some embodiments, the function or property is the level of protein expression (e.g., the expression level of a given gene). In some embodiments, the function or property of the polypeptide is stability (e.g., thermostability measured by thermal denaturation or chemical stability measured by chemical denaturation). In some embodiments, the function or property of the polypeptide is aggregation of the polypeptide.

In some embodiments, more than one assay is performed on the same instrument (e.g., 2 or more, 3 or more, 4 or more, or 5 or more assays). Multiple assays may be performed simultaneously or sequentially on the same instrument. This provides an advantage of simultaneously assaying an entire library of polypeptides with high efficiency. For example, the method may include a determination of competitive binding to a target in the presence of a competitive molecule; measuring binding to multiple different targets; measuring equilibrium binding and binding kinetics; measuring binding and protein stability; or any combination thereof. The present methods may also include assaying multiple functions or properties of each polypeptide under varying conditions, e.g., binding under multiple pH conditions; binding under multiple temperature conditions; and/or binding under multiple buffer conditions.

Exemplary assays of properties or functions of polypeptides are provided in Table 1. One or more of these assays may be performed on the same library of polypeptide. Where more than one assay is performed, the assays may be performed simultaneously or sequentially.

TABLE 1
Assays for properties or functions of polypeptides

| | | Property | |
|---|---|---|---|
| Property | | being | Exemplary |
| or function | Assay | measured | Reference |
| Binding | Ligand | Limit of | Armbruster, |
| | binding | Detection | David A., and |
| | | (LoD) | Terry Pry. |
| | | or Limit of | “Limit of blank, |
| | | Quantitation | limit of detection |
| | | (LoQ) | and limit of |
| | | | quantitation.” |
| | | | The clinical |
| | | | biochemist |
| | | | reviews |
| | | | 29. Suppl 1 |
| | | | (2008): S49. |
| | Equilibrium | Equilibrium | Hulme, Edward |
| | binding | binding | C., and Mike A. |
| | | constant | Trevethick. |
| | | (KD) | “Ligand binding |
| | | | assays at |
| | | | equilibrium: |
| | | | validation and |
| | | | interpretation.” |
| | | | British journal of |
| | | | pharmacology |
| | | | 161.6 (2010): |
| | | | 1219-1237. |
| | Kinetic | binding on | Rich, Rebecca |
| | binding | rate (kon) | L., and David G. |
| | | and/or off | Myszka. |
| | | rate (koff) | “Survey of the |
| | | | year 2007 |
| | | | commercial |
| | | | optical biosensor |
| | | | literature.” |
| | | | Journal of |
| | | | Molecular |
| | | | Recognition: An |
| | | | Interdisciplinary |
| | | | Journal 21.6 |
| | | | (2008): 355-400. |
| | Competitive | Half-maximal | Cox, Karen L., |
| | binding | inhibitory | et al. |
| | | concentration | “Immunoassay |
| | | (IC50), half- | methods.” Assay |
| | | maximal | Guidance |
| | | effective | Manual |
| | | concentration | [Internet]. |
| | | (EC50), or | Eli Lilly & |
| | | inhibition | Company and |
| | | constant (Ki) | the National |
| | | | Center for |
| | | | Advancing |
| | | | Translational |
| | | | Sciences, 2019. |
| Enzymatic | Enzyme | Maximum rate | Robinson, Peter |
| activity | activity | of reaction | K. “Enzymes: |
| | | (Vmax), | principles and |
| | | Michaelis | biotechnological |
| | | constant (Km), | applications.” |
| | | turnover | Essays in |
| | | number (Kcat), | biochemistry 59 |
| | | Catalytic | (2015): 1-41. |
| | | efficiency | |
| | | (Kcat/Km) | |
| | Enzyme | Half-maximal | Copeland, |
| | inhibition | inhibitory | Robert A. |
| | | concentration | Evaluation of |
| | | (IC50), half- | enzyme |
| | | maximal | inhibitors in |
| | | effective | drug discovery: |
| | | concentration | a guide for |
| | | (EC50), | medicinal |
| | | or inhibition | chemists and |
| | | constant (Ki) | pharmacologists. |
| | | | John Wiley & |
| | | | Sons, 2013. |
| Stability | Protein | Thermal | Sancho, Javier |
| | thermal | denaturation | “The stability of |
| | denaturation | midpoint (Tm) | 2-state, 3-state |
| | | | and more-state |
| | | | proteins from |
| | | | simple |
| | | | spectroscopic |
| | | | techniques . . . |
| | | | plus the structure |
| | | | of the |
| | | | equilibrium |
| | | | intermediates at |
| | | | the same time.” |
| | | | Archives of |
| | | | biochemistry |
| | | | and biophysics |
| | | | 531.1-2 (2013): |
| | | | 4-13. |
| | Protein | Chemical | Sancho, Javier. |
| | chemical | denaturation | “The stability of |
| | denaturation | midpoint (Cm) | 2-state, 3-state |
| | | | and more-state |
| | | | proteins from |
| | | | simple |
| | | | spectroscopic |
| | | | techniques . . . |
| | | | plus the structure |
| | | | of the |
| | | | equilibrium |
| | | | intermediates at |
| | | | the same time.” |
| | | | Archives of |
| | | | biochemistry |
| | | | and biophysics |
| | | | 531.1-2 (2013): |
| | | | 4-13. |

### High-Throughput Sequencing of DNA on Beads

Methods for high-throughput determination of the sequence of large pluralities of DNA variants displayed on beads is described herein. The methods described herein can allow high-throughput analysis of proteins in large pluralities of protein-DNA-bead conjugates on one automated instrument as the sequencing of the DNA in said protein-DNA-bead conjugates. In other embodiments, the methods can be used for high-throughput protein analysis and high-throughput sequencing on one automated instrument. In still other embodiments, the plurality of peptide-displaying beads are loaded and immobilized on a solid surface prior to sequencing. Sequencing of large pluralities of DNA variants displayed on protein-DNA-bead conjugates can be achieved using high-throughput sequencing methods and technologies (e. g., sequencing by synthesis (e.g., ILLUMINA™ dye sequencing, ion semiconductor sequencing, or pyrosequencing) or sequencing by ligation (e.g., oligonucleotide ligation and detection (SOLiD™) sequencing or polony-based sequencing), long-read or single-molecule sequencing (e.g., Helicos™ sequencing, single-molecule real-time (SMRT™) sequencing, and nanopore sequencing) and Sanger sequencing)). In yet other embodiments, high-throughput sequencing is achieved via fluorescence detection of incorporated bases on each immobilized bead (sequencing by synthesis).

### Single-Instrument Sequencing of Polynucleotides and Assaying of Polypeptides

Single-instrument sequencing and assaying of polynucleotides, as described herein, can start with introducing protein-DNA-bead conjugates into an instrument (e.g., into microwells or randomly arrayed onto a flow-cell surface). In some embodiments the sequencer/analyzer instrument can be configured to include the following components: a flow-cell to (1) immobilize beads allowing the analysis at a single bead level and to (2) introduce liquid phase reagents in an automated manner; and a high-throughput mechanism to measure signals for both sequencing and protein assays (e.g., automated fluorescence microscopy instrument) where fluorescence signals from sequencing and binding are recorded across all beads. In some embodiments, sequencing and/or binding events produce a change in pH that is detected across all beads, for example as described in U.S. Pat. No. 8,936,763, herein incorporated by reference in its entirety.

In some embodiments varying concentrations of reagents are introduced into the sequence and analysis instrument and the fluorescence or pH signals report the binding of the reagents to the protein-DNA-bead conjugates. Following protein and/or polypeptide assaying, in some embodiments, the sequencing of the DNA encoding the protein is performed by stripping the complementary strand of the DNA (e.g., formamide or NaOH), removing the linked protein, and leaving a plurality of clonal single-stranded DNA (ssDNA) molecules bound to the bead. A primer can then be annealed to the ssDNA molecule and sequencing can be performed (e.g., sequencing-by-synthesis or sequencing by ligation) to determine the sequence of the DNA and the identity of the assayed protein. In some embodiments, assaying a protein and sequencing of the protein-encoding DNA can be performed in any order. In some embodiments, DNA sequencing is performed first and can require that a pre-annealed primer is present prior to the start of the sequencing process.

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. Parallel Identification and Functional Characterization of a Library of Polypeptides on a Single Instrument

A library of approximately 3×10⁷ beads was produced by conjugating each bead to a DNA molecule encoding a polypeptide (Example 1, Step a). As described in detail herein, DNA-linked beads were produced by PCR-amplifying each nucleic acid molecule where one primer is bead-linked to produce a homogeneous population of approximately 10⁵ copies of the nucleic acid molecule on each bead. Each bead was identified by single-base sequencing by incorporation of a fluorophore into the nucleic acid sequence (Example 1, Step b). The polypeptide encoded by the nucleic acid on each bead was expressed by cell-free transcription and translation and the resulting polypeptide was subsequently conjugated to the bead in an enzymatic reaction catalyzed by Sortase A (Example 1, Step c). Each bead, in parallel, was (1) identified by the sequence of the nucleic acid molecule conjugated to the bead; and (2) assayed to determine the binding of the conjugated polypeptide to a fluorescently-labeled antibody; where the identification by sequence and the functional characterization was performed on a single instrument (Example 1, Step d).

The present example demonstrates the ability to link the binding properties of each polypeptide to the sequence of the nucleic acid molecule encoding the polypeptide, thereby determining the identity and the binding function of each polypeptide of the plurality of polypeptides in parallel on the same instrument. The present example is not meant to limit what the inventors consider to be the scope of the present invention. The order of steps, methods of nucleic acid identification, and/or methods of functional characterization of the polypeptides may be modified according to the methods described herein and based on the knowledge of one of skill in the art.

### Materials and Reagents

### DNA Oligonucleotides

Gene blocks (gBlocks) and oligonucleotides (oligos) used in the methods herein described are provided in Table 2.

TABLE 2
List of oligonucleotides used for expressing
polypeptide epitopes.

| Name | | |
|---|---|---|
| (SEQ ID | | Modifi- |
| NO.) | Nucleic acid sequence | cation |
| 3x- | GGGCTACTACTATAATACGACTCACTATAGGGT | None |
| OKmFLAG | AAGTGTGGAAGGAGATATACATATGGATTATAA | |
| (SEQ ID | ATTAGATGATGGCGATTACAAGCTCGACGATAT | |
| NO: 14) | TGACTATAAACTGGATGACGACAAGGGTTCCGG | |
| | AAGTTACCCTTATGATGTGCCTGACTATGCCGGA | |
| | TCTGGCAGTGATTATAAACTCGATGATGGAGAC | |
| | TATAAATTAGACGACATCGACTATAAACTGGAC | |
| | GACGACAAGGGGTCCGGCTCGTTACCTGAAACA | |
| | GGATGATGAGCGGGCCGCAGGGTTTTTTGCTGC | |
| | CGTATGACTCATATGC | |
| 3x- | GGGCTACTACTATAATACGACTCACTATAGGGT | None |
| super- | AAGTGTGGAAGGAGATATACATATGGATTATAA | |
| FLAG | AGATGAAGATGGAGACTACAAAGACGAAGACA | |
| (SEQ ID | TTGACTACAAAGACGAGGACCTTCTCGGGAGTG | |
| NO: 15) | GTTCTTATCCTTACGATGTGCCCGACTACGCCGG | |
| | GAGCGGCTCAGATTACAAAGATGAGGACGGAG | |
| | ATTACAAAGATGAAGATATTGACTATAAAGACG | |
| | AAGATCTCTTAGGGTCCGGCTCGTTACCTGAAAC | |
| | AGGATGATGAGCGAGCCGCAGGGTTTTTTGCTG | |
| | CCGTATGACTCATATGC | |
| 3x- | GGGCTACTACTATAATACGACTCACTATAGGGT | None |
| wtFLAG | AAGTGTGGAAGGAGATATACATATGGATTATAA | |
| (SEQ ID | AGATCATGATGGTGATTACAAGGACCATGATAT | |
| NO: 16) | CGACTATAAAGACGACGACGACAAGGGATCGG | |
| | GTAGCTATCCATATGACGTGCCGGACTATGCTG | |
| | GATCAGGCAGTGACTATAAAGACCACGATGGCG | |
| | ACTACAAAGACCACGACATCGATTACAAAGACG | |
| | ACGACGATAAAGGGTCCGGCTCGTTACCTGAAA | |
| | CAGGATGATGAGCGCGCCGCAGGGTTTTTTGCT | |
| | GCCGTATGACTCATATGC | |
| Sortase | GGGCTACTACTATAATACGACTCACTATAGGGT | None |
| A | AAGTGTGGAAGGAGATATACATATGAAGAAGTG | |
| (SEQ ID | GACCAACCGTCTGATGACGATCGCTGGTGTGGT | |
| NO: 17) | ACTGATCCTGGTAGCAGCATATCTGTTCGCTAAA | |
| | CCACATATCGATAACTACCTGCACGATAAAGAT | |
| | AAGGATGAAAAGATCGAACAATACGATAAAAA | |
| | CGTAAAGGAACAGGCAAGTAAAGATAAAAAGC | |
| | AGCAGGCTAAGCCTCAAATCCCGAAAGACAAGT | |
| | CGAAAGTGGCAGGTTACATCGAAATCCCAGATG | |
| | CTGATATCAAAGAACCAGTATACCCAGGTCCAG | |
| | CAACGCCTGAACAACTGAATCGTGGTGTAAGCT | |
| | TCGCAGAAGAAAACGAAAGTCTGGATGATCAAA | |
| | ATATTAGCATTGCAGGCCACACTTTCATTGACCG | |
| | TCCGAACTATCAATTTACAAATCTGAAAGCAGC | |
| | AAAGAAAGGTAGTATGGTGTACTTCAAAGTTGG | |
| | TAATGAAACACGTAAGTATAAAATGACCAGCAT | |
| | TCGTGATGTTAAACCTACAGATGTTGGTGTTCTG | |
| | GATGAACAAAAGGGTAAAGATAAACAACTGAC | |
| | ACTGATCACTTGTGATGATTACAATGAAAAGAC | |
| | AGGTGTATGGGAAAAACGTAAGATCTTCGTGGC | |
| | AACCGAGGTCAAGTGATAGCATAACCCCTTGGG | |
| | GCCTCTAAACGGGTCTTGAGGGGTTTTTTGCTGC | |
| | CGTATGACTCATATGC | |
| Bead_FP | GGGCTACTACTATAATACGACTCACTATAGGG | None |
| (SEQ ID | | |
| NO: 18) | | |
| bt-Bead_ | GGGCTACTACTATAATACGACTCACTATAGGG | 5′ |
| FP | | Biosg |
| (SEQ ID | | |
| NO: 19) | | |
| Bead_RP | GCATATGAGTCATACGGCAGCAAAAAACCCTGC | None |
| (SEQ ID | GGC | |
| NO: 20) | | |
| AF647- | GCATATGAGTCATACGGCAGCAAAAAAC | 5′ |
| Bead_RP | | Alexa |
| (SEQ ID | | Fluor |
| NO: 21) | | 647 |
| DBCO- | GCATATGAGTCATACGGCAGCAAAAAACCCTGC | 5′ |
| Bead_RP | GGC | DBCO// |
| (SEQ ID | | iSp18 |
| NO: 22) | | |
| Bead_ | GCTCATCATCCTGTTTCAGGTAACGAGCCGGACC | None |
| up- | | |
| stream- | | |
| RP | | |
| (SEQ ID | | |
| NO: 23) | | |

### Peptides

The following peptide was used in the methods described herein.
GLSSK-N3 synthesized by CPC Scientific (Sunnyvale, Calif., USA)

### Buffers

The following buffers were used in the methods herein described.
Streptavidin Binding Buffer (SABB): 1M NaCl, 5 mM Tris pH 8, 1 mM EDTA, 0.05%
Tween-20
TNaTE: 140 mM NaCl, 10 mM Tris pH 8, 0.05% Tween-20, 1 mM EDTA
Phosphate buffered saline (PBS): 1×PBS pH 7.4
TE: 10 mM Tris, 1 mM EDTA pH 7.2
10× Sortase Buffer: 500 mM Tris pH 8, 100 mM CaCl₂), 1.5M NaCl
Antibody binding buffer (ABB): 10 mM Tris pH 8, 140 mM NaCl, 2 mM MgCl₂, 5 mM KCl, 0.02% Tween-20
Incubation Buffer: 1×PBS pH 7.4, 10 mM MgCl₂, 0.02% (v/v) Tween-20, 0.01% (w/v) bovine serum albumin (BSA)

### Sequencing Nucleotides

The following custom dideoxynucleotides (ddNTPs) were used in the methods herein described.
7-Propargylamino-7-deaza-ddATP-ATTO-425
7-Propargylamino-7-deaza-ddGTP-Cy5
5-Propargylamino-ddCTP-ATTO-647N
5-Propargylamino-ddUTP-DY-480XL

### In Vitro Transcription Translation (IVTT Mix)

The following IVTT mix was used in the methods herein described.
PURExpress® In Vitro Protein Synthesis Kit (New England Biolabs (NEB), Ipswich, Massachusetts, USA)

### DNA Polymerases

The following polymerases were used in the methods herein described.
Bsm DNA Polymerase, Large Fragment (ThermoFisher Scientific. Waltham, Massachusetts, USA)
Therminator DNA Polymerase (NEB. Ipswich, Mass., USA)
Sequenase Version 2.0 DNA Polymerase (ThermoFisher Scientific. Waltham, Massachusetts, USA)
Phire HotStart II DNA Polymerase (ThermoFisher Scientific. Waltham, Mass., USA)
   Step a. Display of DNA on Beads

DNA-linked beads were produced by PCR amplification of each nucleic acid molecule (Table 2) where one primer is bead-linked to produce a homogeneous population of approximately 10⁵ nucleic acid molecules on each bead. The beads were divided into three tubes, each tube containing a different polypeptide-coding DNA template. The compartmentalization in separate tubes is analogous to compartmentalizing each bead in a microemulsion. After PCR, this resulted in a population of approximately 3×10⁷ beads, each displaying one of the three polypeptide-coding templates. This tube-compartmentalized PCR on beads may also be accomplished using a microemulsion-compartmentalized PCR to generate many unique sequences displayed on beads, according to methods known to those of skill in the art. A flow cytometer was used to sequence the DNA with reading one base of sequence through single-based extension. A theoretical maximum of 4 polypeptides (identified by A, C, T, or G on the single base read) could be read using the flow cytometer. Three unique sequences were displayed on each bead of the plurality of beads. Expansion of the throughput for characterizing large populations of unique proteins can be achieved using existing sequencing platforms and microemulsion methods known to a person of skill in the art.

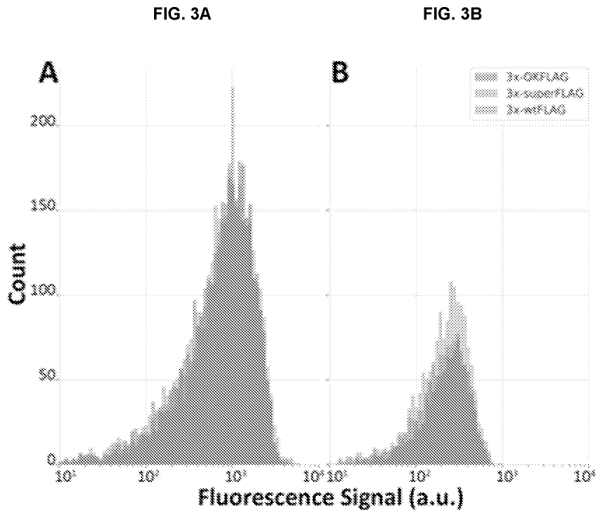

Specifically, three oligonucleotides encoding functionally distinct FLAG peptide epitopes (3×-OKFLAG, 3× wtFLAG, and 3×-superFLAG) were PCR amplified using Phire HotStart II polymerase in separate reaction vials containing standard buffer and 1 μM of primers bt-Bead FP and AF647-Bead_RP. These gene blocks were subjected to thermocycling conditions (98° C. for 2 minutes; followed by 18 cycles of 98° C. for 15 seconds, 57° C. for 15 seconds, and 72° C. for 30 seconds; followed by a final 2-minute extension at 72° C.). Ligation-ready reverse primer was prepared by incubating 40 μM of DBCO-Bead_RP with a 40× excess (1.6 mM) of GLSSK-N3 peptide overnight at room temperature in PBS buffer to yield GLSSK-BA RP. The purified PCR products of 3×-OKFLAG, 3×-wtFLAG, and 3×-superFLAG were separately incubated with −10⁷ Dynabeads® MyOne Streptavidin C1 microspheres (ThermoFisher Scientific, Waltham, Mass., USA) at 500 μM in 25 μL SABB for 30 minutes at room temperature. Beads from the previous step were then washed twice with SABB and resuspended in TNaTE. An aliquot of beads was then analyzed via flow cytometry to confirm DNA capture via high signal in the APC (660±20 nm) channel upon excitation with red laser (618 nm, FIG. 3A). All beads were then washed consecutively with the following to remove the Alexa Fluor 647-labeled anti-sense DNA strand:
1. PBS (one wash)
2. TNaTE (one wash)
3. 20 mM sodium hydroxide (NaOH, three washes)

Washed beads were then suspended in TNaTE and removal of the reverse strand was confirmed via flow cytometry (FIG. 3B). Populations are indistinguishable from uncoated beads, confirming removal of the second strand. At this point, three separate populations of beads display clonal populations of ssDNA encoding their respective FLAG epitope (3×-OKFLAG, 3×-wtFLAG, 3×-superFLAG). The beads were spatially isolated in a manner similar to how they would be during emulsion PCR.

Step b. Single-Base Sequencing of DNA on Beads

Beads displaying three DNA templates encoding three variants of the FLAG peptide in the coding region (3×-OKFLAG, 3× wtFLAG, and 3×-superFLAG) were then prepared for sequencing-by-synthesis. The DNA templates were specifically designed to differ in sequence at the nucleotide immediately following the sequencing primer hybridization site. A flow cytometer was used as the DNA sequencer limiting the reading throughput to a single base. After single-base extension with different fluorescently-labeled nucleotides (ATTO647N-ddCTP, Cy5-ddGTP, and DY480XL-ddUTP), the beads were prepared to be read by the cytometer to distinguish the sequence of the DNA on the beads based on the fluorescence signal in different channels.

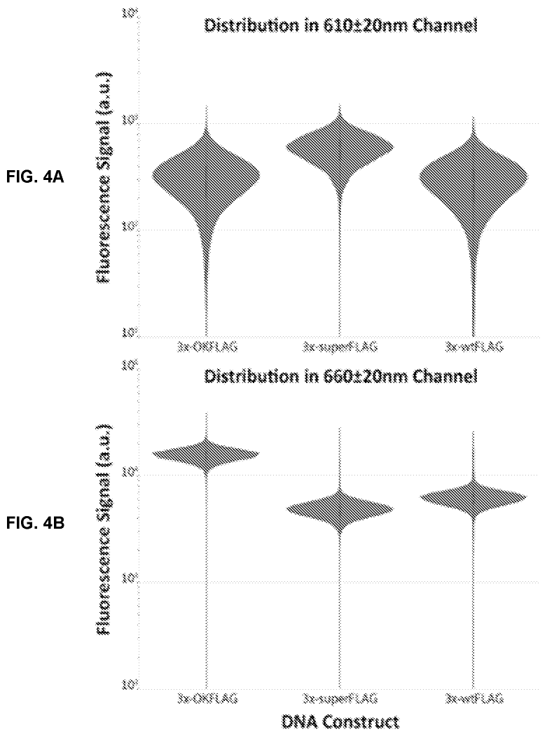

DNA oligos were designed to differ from one another by a single base immediately upstream of the Bead_RP (see underlined base for 3×-OKFLAG, 3×-wtFLAG, and 3×-superFLAG in Table 2). Thus, the identity of the DNA can be determined by identifying which modified ddNTP is displayed on each bead after sequencing. Specifically, incorporation of ddGTP indicates a cytosine (C) on the complementary (sense) strand, incorporation of ddUTP indicates an adenosine (A) on the sense strand, incorporation of ddCTP indicates a guanosine (G) on the sense strand, and incorporation of ddATP indicates a thymine (T) on the sense strand. Beads displaying clonal populations of ssDNA encoding their respective FLAG epitope were washed once with 100 uL SABB and resuspended in 20 μL of SABB containing 500 nM of GLSSK-BA_RP. Then the beads were incubated with 500 nM of GLSSK-BA_RP in 20 uL SABB, heated to 63° C. for 45 s, and flash cooled on ice. Then the beads were washed with 50 μL of 1× Therminator buffer and suspended in 50 μL of cold Jena Sequencing Buffer containing 1× Therminator (Sigma Aldrich) buffer, 1 μM/ea Jena ddNTPs, 10 nM of GLSSK-RP, 0.032 U/μL of Bsm Enzyme (Fisher Scientific) and 0.008 U/μL of Therminator enzyme (Sigma Aldrich). Then the beads were heated to 65° C. for 5 minutes, 63° C. for 20 minutes, and cooled on ice. At this point, the beads were physically separated into three populations, each clonally displaying one of three DNA sequences (3×-OKFLAG, 3×-wtFLAG, or 3×-superFLAG) encoding a FLAG epitope and a terminated nucleotide whose attached fluorophore dictates which epitope is displayed. This step did not require spatial isolation via microemulsions as each bead only picked up a fluorophore-labelled ddNTP that is dependent on the DNA sequence already displayed. Specifically, 3×-OKFLAG recruited ATTO647N-ddCTP (644/669 nm excitation/emission), 3×-wtFLAG recruited Cy5-ddGTP (647/665 nm excitation/emission), and 3×-superFLAG recruited DY480XL-ddUTP (500/630 nm excitation/emission). While ATTO647N and Cy5 have similar fluorescence spectra, the FACS instrument is sensitive enough to distinguish one from another based on the relative intensities in the APC channel (FIGS. 4A and 4B).

Step c. Covalent Attachment of Peptides to Encoding Gene on DNA-Coated Beads

Expression of the bead-conjugated DNA molecules to produce polypeptides was accomplished using IVTT followed by the covalent conjugation of the produced polypeptides to the bead-conjugated DNA molecules with Sortase A. To establish this linkage, the nucleic acid molecules on the beads have a 5′-GLSSK peptide that is the capture moiety (with a free N-terminal glycine), and the polypeptides are genetically encoded in the DNA with an N-terminal LPETG sequence that is the linkage tag. Analogous to dividing the beads into a second microemulsion compartmentalization, the beads were compartmentalized into three separate tubes, each containing the three different DNA constructs. In these tubes, IVTT expression of the bead-linked DNA produces polypeptide which is linked by Sortase A to the nucleic acid, yielding beads linked to both DNA. Sortase A was encoded by exogenous DNA added to the IVTT reaction to produce the enzyme concurrently with the polypeptide.

For compatibility with biological machinery during IVTT, the DNA of a bead population containing partially double-stranded DNA encoding their respective polypeptide epitopes must be made fully double-stranded through annealing and extending an upstream reverse primer. Beads were extended for 20 minutes at 60° C. in buffer containing 1×Bsm buffer, 250 μM/ea dNTPs, 500 nM Bead upstream-RP, and 0.06 U/μL Bsm enzyme. Then the beads from were washed twice with TNaTE and once with water. Then the beads were resuspended in 10 μL of NEB PURExpress® In Vitro Protein Synthesis mix (IVTT mix) following manufacturers protocols and incubated at 37° C. for 2 hours. dsDNA (200 ng) encoding Sortase A was added to 20 μL of NEB IVTT mix and incubated at 37° C. for 2 hours. After incubation, 4 μL of Sortase IVTT mix were added to 10 μL of each bead IVTT mix. 10× sortase buffer (1.55 μL) was added to each tube (three tubes total) and incubated overnight at 4° C. Then beads are spatially separated in different tubes.

Step d. Parallel Determination of Sequence and Binding Activity of Discrete Peptide Epitopes Displayed on DNA-Coated Beads

A binding assay was performed on the population of beads displaying polypeptides and nucleic acids. Beads that were previously compartmentalized (to facilitate faithful display of polypeptide on identifying DNA) were mixed and subjected to a binding incubation with a series of concentrations of peptide-binding antibody. The antibody had varying affinities for the bead-displayed polypeptides. The beads, displaying DNA with a fluorescently incorporated base (sequencing by synthesis) and polypeptide bound to fluorescently-labeled antibody (assay of polypeptide binding function) are then put on the sequencing instrument, here a flow cytometer, in order to read the sequence and the binding of each bead on the same instrument.

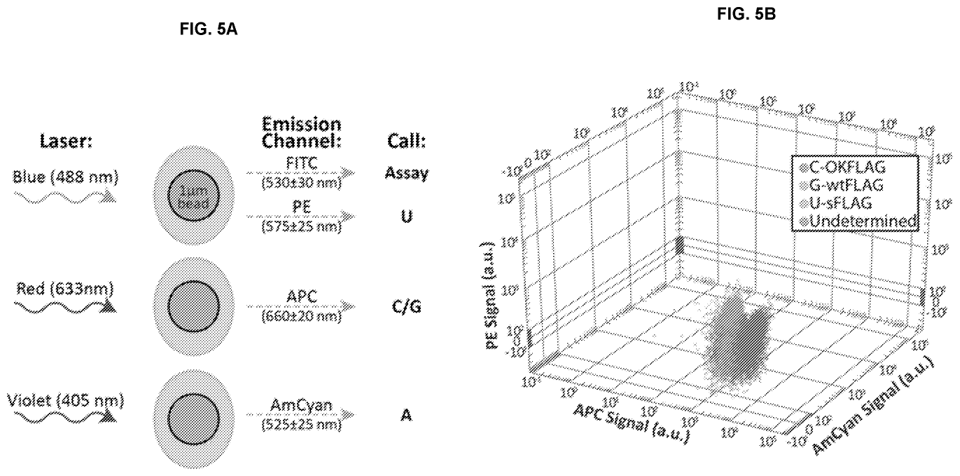

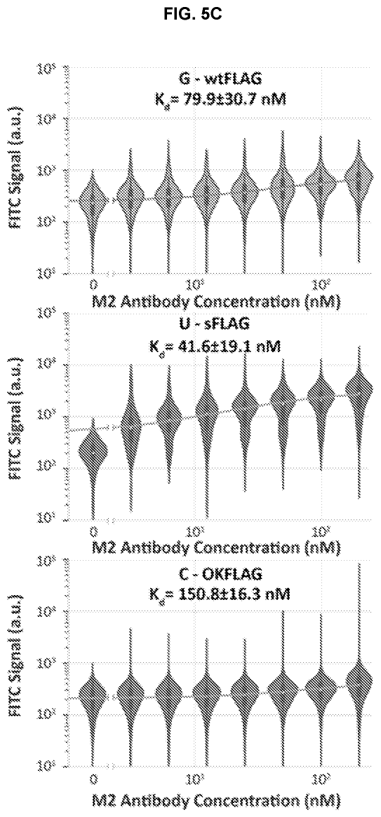

To determine the sequence and binding activity of discrete peptide epitopes on DNA-coated beads a washing step (repeated 2×) with Incubation Buffer and resuspension in Incubation Buffer is performed to remove spent IVTT mix and any non-covalently-attached polypeptides. Then three bead populations were mixed at equal ratios in a new tube. FITC-labelled M2 anti-FLAG antibody (ThermoFisher Scientific. Waltham, Mass., USA) was diluted in incubation buffer and a 1:2 dilution series was prepared containing the following concentrations of M2 anti-FLAG antibody: 200 nM, 100 nM, 50 nM, 25 nM, 12.5 nM, 6.25 nM, 3.125 nM and 0 nM (no target control). Then the bead mixture was split into 8 tubes, the supernatant removed, and 100 uL of M2 anti-FLAG antibody dilution series at the given concentrations was added to each tube. Then the beads were incubated for one hour at room temperature. The beads then underwent two 15 minute washes using 100 uL of PBS and were resuspended in 200 uL of PBS and were assayed using a flow cytometer (FIGS. 5A-5C). At this point, each bead assayed using flow cytometry had a fluorescence value associated with it in each of 15 possible excitation/emission channels. The distribution of values from all beads across these channels allowed us to ascertain with high certainty which FLAG epitope each bead displayed. Then, we gated these beads and plot trends of these discrete populations across various concentrations of the FITC-labelled M2 anti-FLAG antibody to ascertain binding characteristics of these epitopes. The fluorescence of each bead across multiple channels was used, where possible, to determine the identity of the incorporated ddNTP and thus the identity of the oligonucleotide and peptide displayed on each bead. Beads containing identical oligonucleotides at identical antibody concentration were aggregated and their mean fluorescent signal was fit to the following equation:

F^{pep}ₘₑₐₙ([T])=F_{bg}+F^{pep}ₘₐₓ*([T]/([T]+K_{d}^{pep}))

where F^{pep}ₘₑₐₙ([T]) is the mean fluorescent signal for the peptide at a given target concentration, [T], F_{bg} is the background fluorescent signal when [T]=0, F^{pep}ₘₐₓ is the maximum fluorescent signal observed for the peptide at full binding saturation, and K_{d}^{pep} is the equilibrium dissociation constant for the peptide. A single mixture of beads displaying one of three possible peptide epitopes was split and incubated at different concentrations of fluorescent anti-FLAG M2 antibody and analyzed using flow cytometry. The fluorescent signals obtained from each bead at each concentration was sufficient to determine the identity of the oligonucleotide displayed on the bead and an accurate equilibrium binding measurement (dissociation constant) was obtained for the peptides displayed on the beads. The accuracy of the biophysical assay is evidenced by its correlation with previously measured affinities for these three peptides.

Methods for generating beads that covalently display a homogenous population of polypeptides, together with a homogenous population of their encoding DNA by a process of two compartmentalized steps: PCR amplification and polypeptide expression and conjugation have been shown. Furthermore, it is demonstrated that, by sequencing the DNA and assaying polypeptide binding of each bead on a single instrument, the binding properties of each polypeptide are linked to the sequence of the nucleic acid molecule encoding the polypeptide, thereby determining both the identity and the binding function of each individual polypeptide on a per-bead basis.

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims. Other embodiments are within the claims.

We claim:

1. A method of high-throughput analysis of a plurality of polypeptides, the method comprising:
(a) providing a plurality of beads, wherein a bead of the plurality of beads is conjugated to a different nucleic acid molecule encoding a polypeptide;
(b) processing the nucleic acid molecule encoding a polypeptide to produce the encoded polypeptide, wherein the bead of said plurality of beads is conjugated to the encoded polypeptide;
(c) assaying the encoded polypeptide to identify one or more properties of the encoded polypeptide;
(d) sequencing the nucleic acid molecule encoding the polypeptide to identify a sequence of the nucleic acid molecule encoding the polypeptide; and
(d) linking the one or more properties of each polypeptide to the sequence of the nucleic acid molecule encoding the polypeptide.

2. The method of claim 1, wherein the encoded polypeptide is conjugated directly to the bead.

3. The method of claim 1, wherein the encoded polypeptide is conjugated to nucleic acid molecule, thereby conjugating the polypeptide to the bead.

4. The method of claim 1, wherein (a) comprises conjugating each bead of the plurality of beads to a nucleic acid molecule, each nucleic acid molecule encoding a polypeptide of the plurality of polypeptides.

5. The method of claim 1, wherein (b) comprises expressing the nucleic acid molecule to produce the polypeptide and conjugating the polypeptide to the bead or conjugating the polypeptide to the nucleic acid molecule.

6. The method of claim 4, wherein step (a) is performed in a first microemulsion droplet.

7. The method of claim 6, wherein step (a) further comprises amplifying each nucleic acid molecule within each microemulsion droplet, thereby producing a homogeneous population of a nucleic acid molecule on each bead.

8. The method of any one of claims 4-7, wherein steps (b) and (c) are performed in a second microemulsion droplet.

9. The method of any one of claims 4-8, wherein step (b) occurs in vitro in a cell free system.

10. The method of any one of claims 1-9, wherein the nucleic acid is DNA, cDNA, or RNA.

11. The method of any one of claims 1-10, wherein the nucleic acid molecule and the polypeptide are conjugated by expressed protein ligation or by protein trans-splicing.

12. The method of any one of claims 1-11, wherein the bead or the nucleic acid molecule is conjugated to a capture moiety and the polypeptide comprises a linkage tag, wherein the capture moiety and the linkage tag are conjugated, thereby conjugating the bead to the polypeptide or conjugating the nucleic acid molecule to the polypeptide.

13. The method of claim 12, wherein conjugation of the capture moiety and the linkage tag is catalyzed by a linking enzyme.

14. The method of claim 13, wherein the linking enzyme is encoded by a second nucleic acid.

15. The method of claim 13, wherein the linking enzyme is an isolated enzyme.

16. The method of claim 13, wherein the linking enzyme is a sortase, a butelase, a trypsiligase, a peptiligase, a formylglycine generating enzyme, a transglutaminase, a tubulin tyrosine ligase, a phosphopantetheinyl transferase, a SpyLigase, or a SnoopLigase,

17. The method of claim 16, wherein:
the linking enzyme is sortase A;
one of the capture moiety or linkage tag comprises a polypeptide which has a free N-terminal glycine residue; and
the other of the capture moiety or linkage tag comprises a polypeptide comprising amino acid sequence LPXTG (SEQ ID NO: 1) where X is any amino acid.

18. The method of claim 16, wherein:
the linking enzyme is butelase-1;
one of the capture moiety or linkage tag comprises a polypeptide comprising the amino acid sequence X₁X₂XX (SEQ ID NO: 2) where X₁ is any amino acid except P, D, or E; X₂ is I, L, V, or C; and X is any amino acid; and
the other of the capture moiety or linkage tag comprises a polypeptide comprising the amino acid sequence DHV or NHV.

19. The method of claim 16, wherein:
the linking enzyme is trypsiligase;
one of the capture moiety or linkage tag comprises a polypeptide comprising amino acid sequence RHXX (SEQ ID NO: 3) where X is any amino acid; and
the other of the capture moiety or linkage tag comprises a polypeptide comprising the amino acid sequence YRH.

20. The method of claim 16, wherein:
the linking enzyme is omniligase;
capture moiety comprises carboxamido-methyl (OCam); and
the linkage tag comprises a polypeptide comprising a free N-terminal amino acid acting as an acyl-acceptor nucleophile.

21. The method of claim 16, wherein:
the linking enzyme is formylglycine generating enzyme;
the capture moiety comprises an aldehyde reactive group; and
the linkage tag comprises a polypeptide comprising the amino acid sequence CXPXR (SEQ ID NO: 4), wherein X is any amino acid.

22. The method of claim 16, wherein:
the linking enzyme is transglutaminase;
one of the capture moiety or linkage tag comprises a polypeptide comprising a lysine residue or a free N-terminal amine group; and
the other of the capture moiety or linkage tag comprises a polypeptide comprising the amino acid sequence LLQGA (SEQ ID NO: 5).

23. The method of claim 16, wherein:
the linking enzyme is a tubulin tyrosine ligase;
one of the capture moiety or linkage tag comprises a polypeptide comprising a free N-terminal tyrosine residue; and
the other of the capture moiety or linkage tag comprises a polypeptide comprising the C-terminal amino acid sequence VDSVEGEEEGEE (SEQ ID NO: 6).

24. The method of claim 16, wherein:
the linking enzyme is a tubulin phosphopantetheinyl transferase;
the capture moiety comprises coenzyme A (CoA); and
the linkage tag comprises a polypeptide comprising the amino acid sequence DSLEFIASKLA (SEQ ID NO: 7).

25. The method of claim 16, wherein:
the linking enzyme is SpyLigase;
one of the capture moiety or linkage tag comprises a polypeptide comprising amino acid sequence ATHIKFSKRD (SEQ ID NO: 8); and
the other of the capture moiety or linkage tag comprises a polypeptide comprising the amino acid sequence AHIVMVDAYKPTK (SEQ ID NO: 9).

26. The method of claim 16, wherein:
the linking enzyme is SnoopLigase;
one of the capture moiety or linkage tag comprises a polypeptide comprising amino acid sequence DIPATYEFTDGKHYITNEPIPPK (SEQ ID NO: 10); and
the other of the capture moiety or linkage tag comprises a polypeptide comprising the amino acid sequence KLGSIEFIKVNK (SEQ ID NO: 11).

27. The method of claim 16, wherein the capture moiety comprises double-stranded DNA and the linkage tag comprises a polypeptide, wherein the capture moiety and the linkage tag form a leucine zipper.

28. The method of claim 27, wherein:
the capture moiety comprises the nucleic acid sequence TGCAAGTCATCGG (SEQ ID NO: 12); and
the linkage tag comprises the amino acid sequence
(SEQ ID NO: 13)
DPAALKRARNTEAARRSRARKGGC

29. The method of any one of claims 1-28, wherein each bead is conjugated to 100 or more copies of the nucleic acid molecule.

30. The method of any one of claims 1-29, wherein each bead is conjugated to 100 or more copies of the encoded polypeptide.

31. The method of any one of claims 1-30, wherein the plurality of beads of step (a) comprises between 1×10⁶ and 1×10¹⁰ beads, wherein each said bead is conjugated to a polypeptide having a unique amino acid sequence.

32. The method of any one of claims 1-31, wherein one or more copies of the polypeptide having a unique amino acid sequence is conjugated to each of two or more beads within the plurality of beads of step (a).

33. The method of claim 32, wherein the one or more copies of the polypeptide having a unique amino acid sequence is conjugated to each of between 2 and 15 beads within the plurality of beads of step (a).

34. The method of any one of claims 1-33, wherein at least one of the one or more functions or properties of each said polypeptide is assayed at a temperature great than 40° C., at a pH greater than 8.0, and/or at a pH less than 6.0.

35. The method of any one of claims 1-34, wherein the function or property of the polypeptide is a biological activity of the polypeptide.

36. The method of any one of claims 1-34, wherein at least one of the one or more functions or properties of the polypeptide is a binding property of the polypeptide.

37. The method of claim 36, wherein the binding property is quantified by a ligand binding assay, an equilibrium binding assay, and/or a kinetic binding assay.

38. The method of any one of claims 1-34, wherein at least one of the one or more functions or properties of the polypeptide is an enzymatic activity of the polypeptide.

39. The method any one of claims 1-34, wherein at least one of the one or more functions or properties of the polypeptide is the stability of the polypeptide.

40. The method of claim 39, wherein the stability of the polypeptide is quantified by thermal denaturation assay, a chemical denaturation assay, or a pH denaturation assay.

41. The method of any one of claims 1-40, wherein (b)(ii) comprises assaying two or more, three or more, four or more, or five or more properties or functions of the polypeptide.

42. The method of claim 41, wherein assaying the two or more, three or more, four or more, or five or more properties or functions of the polypeptide is performed simultaneously or sequentially.

43. The method of any one of claims 1-42, wherein at least one of the functions or properties is assayed at multiple temperatures, at multiple pH levels, in multiple salt concentrations, and/or in multiple buffers.

44. The method of any one of claims 1-43, wherein the plurality of polypeptides comprises a library of antigens, antibodies, enzymes, substrates, or receptors.

45. The method of claim 44, wherein the library of antigens comprises viral protein epitopes for one or more viruses.

46. A method of conjugating a polypeptide to a bead, the method comprising:
(a) conjugating a nucleic acid molecule encoding the polypeptide to a bead in a first microemulsion droplet; and
(b) processing the nucleic acid molecule in a second microemulsion droplet, wherein processing comprises:
(i) expressing the nucleic acid molecule to produce the polypeptide; and
(ii) conjugating the polypeptide to the nucleic acid molecule.

47. The method of claim 46, wherein conjugation of the polypeptide to the nucleic acid molecule is catalyzed by a linking enzyme.

48. The method of claim 46, wherein the polypeptide is conjugated to the nucleic acid molecule by expressed protein ligation or by protein trans-splicing.

49. The method of claim 46, wherein the polypeptide is conjugated to the nucleic acid molecule by formation of a leucine zipper.

50. The method of claim 46, wherein (a) further comprises amplifying the nucleic acid molecule within the first microemulsion droplet, thereby producing a clonal population of the nucleic acid molecule on the bead.

51. The method of any one of claims 46-50, wherein (b)(i) occurs in vitro in a cell free system.

52. The method of any one of claims 46-51, wherein the nucleic acid is DNA, cDNA, or RNA.

53. The method of any one of claim 46-52, wherein conjugation of the polypeptide to the nucleic acid molecule in step b(ii) is catalyzed by a linking enzyme.

54. The method of any one of claims 46-53, wherein the linking enzyme is encoded by a second nucleic acid.

55. The method of any one of claims 46-54, wherein the linking enzyme is an isolated enzyme.

56. The method of any one of claim 46-55, wherein the linking enzyme is a sortase, a butelase, a trypsiligase, a peptiligase, a formylglycine generating enzyme, a transglutaminase, a tubulin tyrosine ligase, a phosphopantetheinyl transferase, a SpyLigase, or a SnoopLigase,

57. The method of any one of claims 46-56, wherein the nucleic acid molecule is conjugated to a capture moiety and the polypeptide comprises a linkage tag, wherein the capture moiety and the linkage tag are conjugated, thereby conjugating the nucleic acid molecule to the polypeptide.

58. The method of claim 57, wherein the linking enzyme catalyzes the conjugation of the capture moiety and the linkage tag, thereby catalyzing the conjugation of the polypeptide to the nucleic acid.

59. The method of claim 57, wherein the capture moiety comprises double-stranded DNA and the linkage tag comprises a polypeptide, wherein the capture moiety and the linkage tag form a leucine zipper.

60. The method of any one of claims 46-52, wherein the polypeptide is conjugated to the nucleic acid molecule in b(ii) by expressed protein ligation or by protein trans-splicing.
